(19) United States
(12) Patent Application Publication
(10) Pub. No.: US 2018/0235934 A1
(43) Pub. Date: Aug. 23, 2018

(54) NORADRENERGIC DRUG TREATMENT OF OBSTRUCTIVE SLEEP APNEA

(71) Applicant: Massachusetts Institute of Technology, Cambridge, MA (US)

(72) Inventors: Chi-Sang Poon, Lexington, MA (US); Gang Song, Malden, MA (US)

(73) Assignee: Massachusetts Institute of Technology, Cambridge, MA (US)

(21) Appl. No.: 15/753,282

(22) PCT Filed: Aug. 18, 2016
(86) PCT No.: PCT/US2016/047556

§ 371 Date: Feb. 17, 2018

Related U.S. Application Data
(60) Provisional application No. 62/206,698, filed on Aug. 18, 2015.

(51) Int. Cl. A61K 31/4164; A61P 11/00; A61K 31/527; A61K 31/55; A61K 31/4745; A61K 31/496; A61K 31/4178; A61K 31/475; A61K 9/28; A61K 9/48

(52) U.S. Cl. CPC A61K 31/4164; A61P 11/00; A61K 31/527; A61K 31/55; A61K 9/4891; A61K 31/496; A61K 31/4178; A61K 31/475; A61K 9/28; A61K 31/4745

(57) ABSTRACT

Aspects of the disclosure relate to methods for treating disorders using agents for promoting hypoglossal motoneuron excitability. In some instances the disorders include obstructive sleep apnea (OSA), cataplexy, attention deficit/hyperactivity disorder (ADHD), attention deficit disorder (ADD) or depression. Related products are also included within the invention.

### RELATED APPLICATION

This application claims the benefit under 35 U.S.C. § 119(e) of U.S. provisional application No. 62/206,698, filed Aug. 18, 2015, which is incorporated by reference herein in its entirety.

### BACKGROUND OF INVENTION

Decline of noradrenergic drive is thought to be a cause of the sleep state-dependent decreases of hypoglossal motoneuron excitability and resultant pharyngeal dilator (genioglossus) muscle activity¹⁻². Paradoxically, activation of α₁-adrenoceptors and/or serotonin (5-HT) receptors on hypoglossal motoneurons reportedly fails to effectively stimulate genioglossus activity during rapid eye movement (REM) sleep as in wakefulness and non-REM sleep, revealing an inherent complexity of afferents integration by hypoglossal motoneurons^{2,4}.

### SUMMARY OF INVENTION

Sleep state-dependent hypotonia of hypoglossal motoneurons innervating the pharyngeal dilator muscles is a key trigger of obstructive sleep apnea (OSA) in at-risk patients. Quite unexpectedly, it has been discovered according to aspects of the invention that disinhibition of central noradrenergic neurons by α₂-adrenoceptor antagonism restores hypoglossal motoneuron excitability and experience-dependent hypoglossal motor learning and memory capacity and that this intervention mitigates obstructive apnea during REM-like sleep. These findings support new therapeutic approaches to diseases such as OSA, for which effective pharmacotherapy is currently lacking.

Thus, in some aspects the invention is a method for treating OSA by administering to a subject having OSA an agent for promoting hypoglossal motoneuron excitability in an effective amount to treat OSA. It has been discovered, quite surprisingly, that stimulation and/or disinhibition of central noradrenergic neurons not only promotes the baseline excitability of hypoglossal motoneurons but allows experience-dependent (i.e., airway obstruction-dependent) facilitation of the excitability above the baseline. This facilitation effect occurs rapidly during airway obstruction and is “remembered” to some extent to avert further obstruction afterward, creating a long term therapeutic effect in OSA patients. Thus, in some embodiments the agent for promoting hypoglossal motoneuron excitability restores experience-dependent hypoglossal motor learning and memory capacity.

The agent for promoting hypoglossal motoneuron excitability in some embodiments is a disinhibitor and/or stimulant of central noradrenergic neurons. In some embodiments the disinhibitor of central noradrenergic neurons is an α₂-adrenoceptor antagonist such as yohimbine. In other embodiments the disinhibitor of central noradrenergic neurons may be an α₂-adrenoceptor subtype A (alpha-2A) antagonist or an α₂-adrenoceptor subtype C (alpha-2C) antagonist. In other embodiments wherein the α₂-adrenoceptor antagonist is selected from the group consisting of Atipamezole, MK-912, RS-79948, RX 821002, [3H]2-methoxy-idazoxan, and JP-1302.

The agent for promoting hypoglossal motoneuron excitability in some embodiments is in a sustained release formulation. The sustained release formulation may be, for instance, designed to release the agent over 6-10 hours or over 8 hours. The agent for promoting hypoglossal motoneuron excitability may be administered to the subject before bedtime.

In some embodiments the agent for promoting hypoglossal motoneuron excitability acts directly on A7 and/or A5 noradrenergic neurons. In other embodiments the agent for promoting hypoglossal motoneuron excitability acts upstream of an A7 and/or A5 neurons by inducing an activator of the A7 and/or A5 neurons. In yet other embodiments the agent for promoting hypoglossal motoneuron excitability acts upstream of an A7 and/or A5 neurons by blocking an inhibitor of the A7 and/or A5 neurons.

The agent for promoting hypoglossal motoneuron excitability may be administered to the subject on any schedule, routine or as needed. For instance, the agent for promoting hypoglossal motoneuron excitability is administered to the subject on a daily basis for 6 months, a daily basis for 3 months, or a daily basis for 1 month. In some embodiments the agent for promoting hypoglossal motoneuron excitability is administered to the subject on a daily basis in several cycles, wherein the subject is not administered the agent for promoting hypoglossal motoneuron excitability for a period of time in between cycles.

The subject may in some embodiments not be coadministered a serotonin receptor antagonist with the agent for promoting hypoglossal motoneuron excitability.

The invention, in other aspects is a composition of a sustained release formulation of yohimbine. The sustained release formulation may be a time release formulation, wherein the yohimbine is released from the formulation at specific time intervals. In other embodiments the sustained release formulation is a layered tablet, having layers of yohimbine in between layers of polymer. In yet other embodiments the sustained release formulation is constructed to release the yohimbine over a 6-10 hour time interval.

In other aspects the invention is a method for treating cataplexy, attention deficit/hyperactivity disorder (ADHD), attention deficit disorder (ADD) or depression by administering to a subject having cataplexy, ADHD, ADD or depression an agent for promoting hypoglossal motoneuron excitability in an effective amount to treat the cataplexy, ADHD, ADD, or depression. In some embodiments the agent for promoting hypoglossal motoneuron excitability is a disinhibitor or stimulant of central noradrenergic neurons.

The invention in other aspects is a transdermal patch comprising a drug reservoir housing an agent for promoting hypoglossal motoneuron excitability, a semi-permeable layer on one side of the drug reservoir, and an impermeable layer on an opposing side of the drug reservoir, wherein the semipermeable layer is arranged to release the agent for promoting hypoglossal motoneuron excitability over a period of 6-10 hours.

In yet other aspects the invention is a composition of a sustained release tablet or capsule comprising an agent for promoting hypoglossal motoneuron excitability and one or more sustained release coatings constructed arranged to release the agent for promoting hypoglossal motoneuron excitability over a period of 6-10 hours. In some embodiments the sustained release tablet or capsule is constructed and arranged to release the agent for promoting hypoglossal motoneuron excitability in a dosage of 3-6 mg per 1-3 hours. In other embodiments the sustained release tablet or capsule is constructed and arranged to release the agent for promoting hypoglossal motoneuron excitability during REM and nonREM sleep.

The agent for promoting hypoglossal motoneuron excitability in some embodiments is an α2-adrenoceptor antagonist. In other embodiments the agent for promoting hypoglossal motoneuron excitability is an α2-adrenoceptor alpha-2A antagonist. In yet other embodiments the agent for promoting hypoglossal motoneuron excitability is an α2-adrenoceptor alpha-2C antagonist. The α2-adrenoceptor antagonist in some embodiments is selected from the group consisting of a yohimbine, Atipamezole, MK-912, RS-79948, RX 821002, [3H]2-methoxy-idazoxan, and JP-1302. The yohimbine optionally is derived from a yohimbe herbal extract.

In some embodiments the composition does not include a serotonin receptor antagonist.

Each embodiment described herein also includes a composition for use, use of the composition as well as a method for manufacturing a medicament for use as described herein.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

These and other aspects of the invention, as well as various embodiments thereof, will become more apparent in reference to the drawings and detailed description of the invention.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C show that activation of α₂-adrenoceptors on pontine A7 and A5 noradrenergic neurons impaired the capacity for experience-dependent learning and memory in hypoglossal motor defense against airway occlusion. FIG. 1A presents tracings of integrated genioglossus EMG and diaphragm EMG recordings in one rat. FIG. 1A upper panel: Microinjection of α₂-adrenoceptor specific agonist clonidine at bilateral A7 region reduced baseline genioglossus activity but not diaphragmatic activity. Episodic airway occlusion treatment (denoted by dots above the integrated genioglossus EMG recording) elicited relatively weak facilitation of genioglossus activity during each occlusion episode with no evidence of long-term facilitation post-treatment. FIG. 1A lower panel: After washout of clonidine, episodic airway occlusion treatment elicited much stronger facilitation of genioglossus activity during each occlusion episode with pronounced long-term facilitation post-treatment. Expanded views of the corresponding integrated genioglossus EMG and diaphragm EMG recordings during the last occlusion episode (area boxed) in each case are shown in FIG. 5. FIG. 1B depicts the average data showing the effects of clonidine injection at bilateral A7 region on baseline GG activity before episodic airway occlusion (left panel) as well as facilitation of GG activity during the first and last airway occlusion episodes (middle panel) and at 5 min and 40 min after the last airway occlusion episode (right panel). Dotted areas indicate the tonic component of GG EMG during the application of airway occlusion. FIG. 1C shows the similar results observed after clonidine microinjection at bilateral A5 region in all rats (n=6) except that baseline GG activity was not significantly reduced in this case. *P<0.05 vs. baseline; † P<0.05 between values as indicated.

FIGS. 2A-2E show that systemic yohimbine reversed the depressions of baseline hypoglossal activity and obstructive apnea-induced hLTF during REM sleep. FIG. 2A shows tracings of integrated hypoglossal nerve and phrenic nerve activities in one rat. FIG. 2A upper panel: Microinjection of carbachol at dorsomedial pons evoked a REM sleep-like state as indicated by the appearance of hippocampal theta discharge and decrease in the amplitude of hypoglossal nerve discharge. Episodic airway occlusion treatment (denoted by dots above the integrated hypoglossal nerve recording) elicited relatively weak facilitation of hypoglossal activity during each occlusion episode with no evidence of long-term facilitation post-treatment. FIG. 2A lower panel: After the induction of REM-like sleep with carbachol, systemic administration of yohimbine by intravenous injection (0.75 mg/kg i.v.) increased the amplitude of hypoglossal nerve discharge to above the pre-carbachol baseline level in one rat. Episodic airway occlusion treatment elicited much stronger facilitation of hypoglossal nerve discharge during each occlusion episode with pronounced long-term facilitation post-treatment. Systemic yohimbine also restored the facilitation of hypoglossal amplitude during and after episodic airway occlusion (cf. FIGS. 8A, 8B). Expanded views of the integrated hypoglossal and phrenic nerve discharge recordings during the last occlusion episode (area boxed) in each case are shown in FIG. 7. FIG. 2B presents average data (n=7) showing the effects of dorsomedial pontine carbachol microinjection and intravenous yohimbine injection on the amplitude of hypoglossal nerve discharge in all rats before, during (first and last episodes) and after (5 min and 20 min) episodic airway occlusion treatment compared with corresponding effects in control conditions (without carbachol and yohimbine). Number of rats in each group control, n=12; carbachol, n=6, carbachol+yohimbine, n=7. *P<0.05 (vs. baseline; broken line); ^{#}P<0.05. FIG. 2C is a replot of the data in FIG. 2B for direct comparison of the amplitude of hypoglossal nerve discharge before and after episodic airway occlusion in the carbachol group (left panel) and carbachol+yohimbine group (right panel). ^{$}P<0.05. FIGS. 2D and 2E are similar to FIGS. 2A and 2B, but with a lower dose of systemic yohimbine (0.3 mg/kg i.v.) under spontaneous REM sleep. *P<0.05, n=5.

FIGS. 3A-3B show noradrenergic A7 and A5 neuronal groups are activated by episodic airway occlusions, as indicated by enhanced c-Fos expression. FIG. 3A: Photomicrographs showing A7 and A5 noradrenergic neurons identified by dopamine β-hydroxylase (DBH) immunofluorescent labeling that were immunopositive to c-Fos in a rat exposed to episodic airway occlusion, compared with one that was not exposed (control). “x” indicates artifacts FIG. 3B: Percentage of c-Fos immunopositive neurons among A7, A5 or A6 noradrenergic neurons in the experimental group (n=6) and control group (n=4). LC, locus coeruleus; SubC, sub-coeruleus. * P<0.01.

FIG. 4 presents photomicrographs showing loci of clonidine injection in A7 and A5 regions. Microinjection loci of clonidine or yohimbine were marked by subsequent microinjection of fluorescent microspheres; the center of microinjection is indicated by the marker (arrow). The noradrenergic neurons of A7 or A5 were revealed using anti-TH (tyrosine hydroxylase) immunostaining. The fluorescent TH-immunopositive neurons in A7 or A5 were within the diffusion range of the microinjection. KF, Kölliker-Fuse nucleus; LC, locus coeruleus; LVPO, lateroventral periolivary nucleus; scp, superior cerebellar peduncle.

FIG. 5 presents expanded views of the boxed areas shown in FIG. 1A. The upper panel of FIG. 5 shows the expanded view of the boxed area in the upper panel of FIG. 1A; the lower panel of FIG. 5 shows the expanded view of the boxed area in the lower panel of FIG. 1A.

FIGS. 6A-6B show yohimbine applied systemically or at bilateral A7 or A5 regions increased hypoglossal activity and enhanced obstructive apnea-induced hLFT. FIG. 6A shows yohimbine administered by microinjections at bilateral A7 and A5 regions (50 nl at 2.5 mM at each injection site, n=5) or systematically by intravenous injection (0.5 mg/kg, n=5) augmented baseline hypoglossal amplitude before episodic airway occlusion (left panel) as well as facilitated hypoglossal amplitude during the first and last airway occlusion episodes (middle panel) and at 5 min and 20 min after the last airway occlusion episode (right panel) compared with control (n=12). See FIG. 4 for microinjection loci.*P<0.05 (vs. pre-injection baseline, broken line); ^{#}P<0.05. FIG. 6B is a replot of FIG. 6A for direct comparison of the amplitude of hypoglossal nerve discharge before and after episodic airway occlusion in the yohimbine microinjection group (left panel) and systemic yohimbine group (right panel). ^{$}P<0.05.

FIG. 7 presents expanded views of the boxed areas shown in FIG. 2A. The upper panel of FIG. 7 shows the expanded view of the boxed area in the upper panel of FIG. 2A; the lower panel of FIG. 7 shows the expanded view of the boxed area in the lower panel of FIG. 2A.

FIGS. 8A-8D demonstrate that episodic obstructive apnea failed to induce hLTF during REM sleep. FIG. 8A shows airway occlusion applied at the end of expiration (lasting 10-15 seconds, denoted by dots above the integrated hypoglossal nerve recording, ∫Hypoglossal) in a urethane-anesthetized, paralyzed and mechanically ventilated rat elicited time-dependent facilitation of the amplitude of hypoglossal nerve activity (which comprised predominantly the inspiratory-phasic component) during each occlusion episode. Such airway occlusion when applied repeatedly for 10-12 episodes (to simulate episodic obstructive apnea) induced sustained facilitation of hypoglossal amplitude (hLTF) afterwards. FIGS. 8B and 8C show that, after microinjection of the cholinergic agonist carbachol at dorsomedial pons to induce a REM-like sleep state in one rat (FIG. 8B) or during spontaneously occurring REM sleep in another rat (FIG. 8C), the baseline hypoglossal amplitude is markedly depressed and airway occlusion elicits relatively weak time-dependent facilitation of hypoglossal amplitude compared with the control state (FIG. 8A) above. Also, episodic airway occlusion no longer induced sustained hLTF. In FIGS. 8B and 8C, the onset of REM sleep is indicated by increased hippocampal activity and decreased baseline hypoglossal activity. FIG. 8D presents summary data showing the effects of cholinergic-induced REM sleep (n=5) and spontaneous REM sleep (n=5) on baseline hypoglossal amplitude before episodic airway occlusion (left panel) as well as facilitation of hypoglossal amplitude during the first and last airway occlusion episodes (middle panel) and at 5 min and 20 min after the last airway occlusion episode (right panel). * P<0.05.

FIGS. 9A-9E show that episodic optogenetic stimulation at the A7 region induces post-stimulation hLTF. FIG. 9A is immunohistological imaging showing that most A7 and A5 neurons that expressed EYFP after transduction with HSV were also immunopositive to the catecholamine marker tyrosine hydroxylase (TH). FIG. 9B shows episodic optical stimulation (10 square-wave light pulses at 1 pulse per minute, each lasting 15 seconds) at the A5 region expressing ChR2-EYFP, which evoked hLTF post-stimulation. FIG. 9C is similar to FIG. 9B, but with episodic optical stimulation at the A7 region. FIG. 9D shows that, after systemic application of the α₁-adrenergic antagonist prazosin, episodic optical stimulation at A7 region no longer evokes post-stimulation hLTF. FIG. 9E is summary data showing corresponding responses during (left panel) and after (right panel) episodic optical stimulation of A7 in 5 rats. Optogenetic data for the HSV and AAV vectors were similar and were merged for statistical analysis. ∫Hypoglossal is normalized relative to baseline (dashed line). * P<0.05 vs. baseline.

FIGS. 10A-10C shows the activation of α₂-adrenoceptors on pontine A7 or A5 noradrenergic neurons impaired obstructive apnea-induced hLTF. FIGS. 10A-10C show tracings of integrated genioglossus electromyogram (GG EMG) in one urethane-anesthetized, spontaneously breathing rat. FIG. 10A shows the microinjection of α₂-adrenoceptor agonist clonidine at bilateral A7 region (˜50 nl at 5 mM at each injection site) reduced baseline GG activity. FIG. 10B shows that episodic airway occlusion (denoted by dots above the integrated GG EMG recording) elicited relatively weak facilitation of GG activity during each occlusion episode with no evidence of long-term facilitation afterward. See FIG. 4 for microinjection loci. FIG. 10C shows that, after washout of clonidine (2-3 hours after the microinjections), baseline GG activity was restored to control levels. Episodic airway occlusion elicited much stronger facilitation of GG activity during each occlusion episode with pronounced long-term facilitation of the inspiratory-phasic component of GG activity afterward. Note that a tonic component of GG activity was recruited during airway occlusion but this tonic component decayed rapidly after each airway occlusion episode and did not exhibit long-term facilitation afterward.

FIG. 11 is a neural network diagram showing the proposed two-tier noradrenergic-dependent mechanism in the pathogenesis of OSA and corresponding mechanism of action of yohimbine therapy for OSA. hLTF, hypoglossal long-term facilitation; VLM, ventrolateral medulla. The present disclosure shows that yohimbine effectively restores the excitatory modulations of hypoglossal motoneuron activity by central noradrenergic drive and hLTF during REM sleep.

FIGS. 12A-12B show BRL44408 treatment of obstructive sleep apnea. FIG. 12A illustrates typical recordings in one rat, showing marked decrease of hypoglossal activity during spontaneous REM-like sleep (as indicated by a corresponding increase of hippocampal activity). BRL44408 (0.2 mg/kg, i.v.) restored baseline hypoglossal activity to normal level, with pronounced facilitation of hypoglossal activity during episodic airway occlusion (arrows) in defense against the obstructive apnea. Note that after episodic airway occlusion hypoglossal activity remained at or above normal baseline level under BRL44408 treatment throughout spontaneous REM-like sleep. FIG. 12B shows summary data for 5 rats. * P<0.05 vs. baseline hypoglossal activity. Values are means±SE.

### DETAILED DESCRIPTION

The present disclosure, in some aspects, includes methods for treating disease by up-regulating the activity of central noradrenergic neurons. It has been proposed that obstructive apnea per se may time-dependently facilitate hypoglossal activity leaving a subsequent long-lasting memory called hypoglossal long-term facilitation, a form of experience-dependent motor learning and memory which requires intermittent interruptions of vagal feedback and α₁-adrenoceptor (but not 5-HT₂ receptor) activation on hypoglossal motoneurons for its induction⁵. However, whether and how this phenomenon may play a role in the pathogenesis of OSA has been unclear. The inventors have demonstrated herein that impairment of such experience-dependent motor defense against recurrent obstructive apneas due to noradrenergic withdrawal during sleep contributes to the pathogenesis of OSA beyond the hypotonia of hypoglossal motoneurons. It was reasoned that reactivation of central noradrenergic drive during sleep with therapeutic agents that target central noradrenergic neurons rather than hypoglossal motoneurons could be more effective in restoring hypoglossal motoneuron excitability and hypoglossal long-term facilitation in defense against OSA.

As shown in the Examples section below, it was first demonstrated according to the invention that central noradrenergic neurons activity was influenced by obstructive apnea. It was found that central noradrenergic neurons of the A7 and A5 groups were involved in the induction of noradrenergic-dependent hypoglossal long-term facilitation. Further, suppression of A7 and A5 neuronal activity by focal injection of the α₂-adrenoceptor selective agonist clonidine at bilateral A7 or A5 regions prior to episodic airway occlusion treatment in an animal model reduced the integrated genioglossus EMG amplitude from pre-injection baseline indicating a decrease in hypoglossal activity. Thus, even partial noradrenergic withdrawal with relatively modest or negligible resultant decreases in genioglossus muscle tone can severely impair the capacity for learning and memory in the hypoglossal motor defense against airway occlusion. In contrast to clonidine, the α₂-adrenoceptor specific antagonist yohimbine when injected at bilateral A7 and A5 regions resulted in an up-regulation of hypoglossal motoneuron excitability and of obstructive apnea-induced hypoglossal long-term facilitation. Additional experiments were performed to determine whether systemic yohimbine could provide an effective remedy for OSA, by administering yohimbine intravenously in urethane-anesthetized rats after focal injection of the cholinergic agonist carbachol at pontine dorsomedial reticular formation to induce a REM sleep-like state. These data suggest that disinhibition of A7 and A5 neurons and other central noradrenergic neurons with yohimbine blockade of α₂-adrenoceptors helps to rescue the experience-dependent hypoglossal motor defense against OSA. Thus, reactivation of central noradrenergic drive with agents for promoting hypoglossal motoneuron excitability such as α₂-adrenoceptor specific antagonist therapy, even without other agents should suffice in mitigating and treating OSA and other disorders.

The methods of the invention, in some aspects relate to the treatment of disorders such as OSA, ADHD, ADD, cataplexy and depression. The methods involve the administration to a subject in need of therapy an agent for promoting hypoglossal motoneuron excitability. An agent for promoting hypoglossal motoneuron excitability, as used herein, is a compound that is capable of promoting norepinephrine (a.k.a. noradrenaline) production by neurons of the central nervous system. The agent for promoting hypoglossal motoneuron excitability may be a disinhibitor of central noradrenergic neurons or it may be a stimulant of central noradrenergic neurons. A disinhibitor of central noradrenergic neurons is an agent that removes or neutralizes an inhibitory signal of a norepinephrine producing neuron of the central nervous system. For instance, a disinhibitor may be an α₂-adrenoceptor antagonist, such as an α₂-adrenoceptor subtype alpha-2A antagonist or an α₂-adrenoceptor subtype alpha-2C antagonist. A stimulant of central noradrenergic neurons is a compound that promotes production of norepinephrine by neurons of the central nervous system. A stimulant may act at excitatory receptors on central noradrenergic neurons or an effector upstream of the central noradrenergic neurons, to produce physiologically effective quantities of norepinephrine.

An α₂-adrenoceptor antagonist, as used herein, is a compound which reduces the activity of an α₂-adrenoceptor. α₂-adrenoceptor's include but are not limited to α₂-adrenoceptor subtype alpha-2A antagonists and α₂-adrenoceptor subtype alpha-2C antagonists. The α₂-adrenoceptor antagonist may be a selective α₂-adrenoceptor antagonist. A selective α₂-adrenoceptor antagonist is a compound which is at least 10 times more effective at antagonizing an α₂-adrenoceptor than any other receptor. In some embodiments the selective α₂-adrenoceptor antagonist is at least 15 times, at least 20 times, at least 30 times, at least 40 times, at least 50 times, at least 100 times, at least 1000 times more effective at antagonizing an α₂-adrenoceptor than any other receptor.

The agent for promoting hypoglossal motoneuron excitability restores experience-dependent hypoglossal motor learning and memory capacity. For instance, such a compound may not only promote the baseline excitability of hypoglossal motoneurons but may also produce experience-dependent (i.e., airway obstruction-dependent) facilitation of the excitability above the baseline. This facilitation effect occurs rapidly during airway obstruction and is “remembered” to avert further obstruction afterward.

One example of an agent for promoting hypoglossal motoneuron excitability is yohimbine, including extracts, salts and derivatives thereof as well as agonists, variants, polymorphs, solvates, enantiomers, stereoisomers and hydrates thereof. Yohimbine is also referred to as yohimbine hydrochloride, Yobinol; ParkiMbine; Tosanpin;Yohydrol;YOHIMBE HCL; Menolysin;(16α,17α)-; QUERBRACHINE antagonil; GyniMbine; and yohimbe. Yohimbine (C₂₁H₂₆N₂O₃) is an indolalkylamine alkaloid derived from the bark of Pausinystalia Yohimba, a tree of the Rubiaceae family, of Corynanthe Yohimba, of Pseudocinchona Yohimba, and from the roots of Rauwolfia Serpentina, isolated from the alcoholic extract thereof with other alkaloids having a similar chemical constitution. Yohimbine is a known potent and selective α₂-adrenoceptor antagonist. An exemplary structure for a yohimbine is:

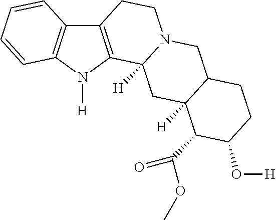 [SMILES: COC(=O)[C@H]1[C@@H](O)CCC2CN3CCc4c([nH]c5ccccc45)[C@@H]3C[C@@H]21]

The α₂-adrenoceptor antagonists useful herein also include Atipamezole (Antisedan from Pfizer), MK-912 (available from Sigma-Aldrich), RS-79948 (available from Tocris), RX 821002 or [3H]2-methoxy-idazoxan (alpha-2A, 2D antagonist—available from Tocris), SKF-86466 Asenapine (USAN, rINNM and BANM; trade names Saphris, Sycrest), JP-1302 (alpha-2C antagonist—available from Tocris), and BRL-44408 as well as extracts, agonists, variants, salts and derivatives thereof and polymorphs, solvates, enantiomers, stereoisomers and hydrates thereof.

BRL-44408 ((2-[2H-(1-Methyl-1,3-dihydroisoindole)methyl]-4,5-dihydroimidazole) acts as a selective antagonist for the α2A adrenoreceptor and has the following chemical structure.

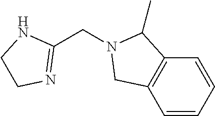 [SMILES: CC1c2ccccc2CN1CC1=NCCN1]

MK-912 (((2S,12bS)1′,3′-Dimethylspiro(1,3,4,5′,6,6′,7,12b-octahydro-2H-benzo[b]furo[2,3-a]quinazoline)-2,4′-pyrimidin-2′-one), Prazosin (P7791), and ARC 239 ((2-[2-[4-(o-Methoxyphenyl)piperazin-1-yl]ethyl]-4,4-dimethyl-1,3-(2H,4H)-isoquinolinedione) available from simga-aldrich are selective α2C antagonists.

Prazosin (P7791), ARC 239 (A5736), Imiloxan (19531), and Rauwolscine available from simga-aldrich are selective α2B antagonists.

Atipamezole has the following chemical structure.

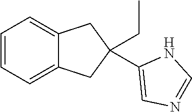 [SMILES: CCC1(c2cnc[nH]2)Cc2ccccc2C1]

RX 821002 (2-(2,3-Dihydro-2-methoxy-1,4-benzodioxin-2-yl)-4,5-dihydro-1H-imidazole hydrochloride) has the following chemical structure.

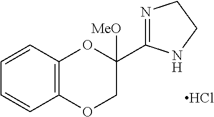 [SMILES: COC1(C2=NCCN2)COc2ccccc2O1 ; Cl]

SKF-86466 (6-Chloro-2,3,4,5-tetrahydro-3-methyl-1H-3-benzazepine) has the following chemical structure.

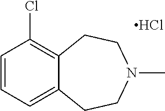

RS-79948 ((8aR,12aS,13aS)-5,8,8a,9,10,11,12,12a,13,13a-dechydro-3-methoxy-12-(ethylsulfonyl)-6H-isoquino[2,1-g][1,6]naphthyridine hydrochloride) has the following chemical structure.

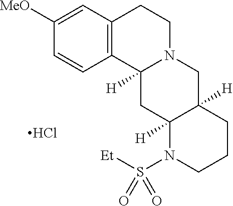 [SMILES: CCS(=O)(=O)N1CCC[C@@H]2CN3CCc4cc(OC)ccc4[C@@H]3C[C@@H]21 ; Cl]

JP-1302 has the following chemical structure.

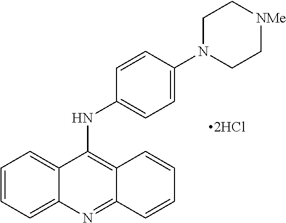 [SMILES: CN1CCN(c2ccc(Nc3c4ccccc4nc4ccccc34)cc2)CC1]

BHT 920 (5,6,7,8-Tetrahydro-6-(2-propenyl)-4H-thiazolo[4,5-d]-azepine-2-amine) and BHT 933 (6-Ethyl-5,6,7,8-tetrahydro-4H-oxazolo[4,5-d]azepin-2-amine) may also be antagonists.

The compounds described herein are useful for treating several disease including OSA, cataplexy, ADHD, ADD and depression. OSA is a highly prevalent sleep disorder, affecting one in five adults in the United States. One in fifteen adults has moderate to severe OSA requiring treatment. Untreated OSA results in reduced quality of life measures and increased risk of disease including hypertension, stroke, heart disease, etc. Continuous positive airway pressure (CPAP) is a standard treatment for OSA. Alternative treatments include oral appliance therapy, upper-airway surgeries and hypoglossal nerve stimulation. Currently there are no effective drug therapies for treatment of OSA.

OSA is characterized by pauses in breathing during sleep. Subjects having OSA stop breathing during sleep numerous times during the night. Typically, OSA is caused by episodes of physical obstruction of the upper airway channel during sleep. The physical obstruction is often caused by changes in the position of the tongue during sleep that results in the closure of the soft tissues at the rear of the throat or pharynx.

Cataplexy is a rare disease, the exact cause of which is unknown. The condition is strongly linked to experiencing intense emotions and reduced levels of the neurotransmitter hypocretin (a.k.a/ orexin). Cataplexy is a sudden and transient episode of loss of muscle tone, often triggered by emotions such as laughter, fear, anger, frustration, annoyance, nervousness, embarrassment, and sadness. The sudden loss of muscle tone in cataplexy is similar to rapid eye movement (REM)-associated muscle atonia during sleep, but it is occurring during wakefulness. A cataplectic attack is sudden in onset and is localized to a specific muscle group or parts of the body.

The methods of the invention are useful for treating a subject in need thereof. A subject in need thereof is a subject that has OSA, cataplexy, ADHD, ADD, or depression. A subject shall mean a human or vertebrate animal or mammal including but not limited to a dog, cat, horse, cow, pig, sheep, goat, turkey, chicken, and primate, e.g., monkey.

In its broadest sense, the terms “treatment” or “to treat” refer to both therapeutic and prophylactic treatments. If the subject in need of treatment is experiencing a condition (i.e., has or is having a particular condition), then “treating the condition” refers to ameliorating, reducing or eliminating one or more symptoms associated with the disorder or the severity of the disease or preventing any further progression of the disease. If the subject in need of treatment is one who is at risk of having a condition, then treating the subject refers to reducing the risk of the subject having the condition or preventing the subject from developing the condition.

Preferably the agents described herein are administered before bedtime or are administered at an appropriate time in a formulation that will release the agents beginning at bed time. The administration prior to bedtime does not imply at night or a particular hour or time of day; rather, before or prior to bedtime means that the composition is preferably administered, generally within about 1 hour prior to a person's normal rest or sleep (typically 4 to 10-hours) period. The absorption half-life is within minutes and elimination half-life is more than half an hour. This dosage administration time will produce the appropriate levels of neuronal activation to treat OSA as described herein.

An effective amount of the compounds of the invention, in some embodiments is that amount that reduces an apnea/hypopnea index to less than 15 events/hour in 50% of adult patients with moderate OSA. In other embodiments an effective amount of the compounds of the invention, in some embodiments is that amount that reduces an apnea/hypopnea index to less than 5 events/hour in 80% of adult and pediatric patients with severe OSA. Patients having moderate versus severe OSA are known to the skilled artisan.

Therapeutic compounds associated with the invention may be directly administered to the subject or may be administered in conjunction with a delivery device or vehicle. Delivery vehicles or delivery devices for delivering therapeutic compounds to surfaces have been described. The therapeutic compounds of the invention may be administered alone (e.g., in saline or buffer) or using any delivery vehicles known in the art.

Yohimbine is a prescription α2-adrenergic blocker which has been tested extensively in acute and long-term clinical studies to verify its relative safety when administered at a clinically recommended dose. Within this dose range yohimbine does not disrupt REM sleep or cause weight gain. For comparison, a yohimbine dose of 0.5 mg/kg presently used in rats amounts to a human equivalent dose of 5.6 mg for a 70 kg person.

The compounds of the invention may be formulated as a sustained release formulation. In view of the short half-life of the antagonists of the invention, it is desirable to administer the compound in a formulation that delivers a therapeutically effective amount over the course of the sleeping time. For instance the half-life of yohimbine is about 36 minutes in humans. In some embodiments the sustained release formulation may be designed to deliver the drug over a 4-10, 4-8, 6-8, 6-10, 7-8, or 8-9 hour window of time. The dose delivered over that period of time may be constant over the whole time period or may vary depending on the anticipated REM-nonREM cycles, the type of drug being administered and it's particular half-life as well as other factors are known to the skilled artisan.

The sustained release formulation of the invention may be any type of sustained release device including, for example, tablets, capsules, and transdermal patches. For instance the transdermal patch may include 1 or more reservoirs housing the drug and a semi permeable membrane that allows for slow release of the drug through the skin. Sustained release transdermal patches are well known in the art.

In some embodiments the compositions of the invention are tablets or capsules having a core and one or more coatings surrounding the core, such as a sustained or delayed release layer. In some embodiments the sustained release layer includes a combination of water-soluble polymers and water-insoluble polymers. The sustained release coating can contain a combination of polyethylene oxide and an ethylcellulose, for example, or a hydroxypropylmethyl cellulose and ethylcellulose. An ethylcellulose product that can be used in the disclosed dosage forms is Ethocel(Dow Chemical Company). The rate of dissolution of the sustained release layer can be controlled by adjusting the ratio of water-soluble polymer to water-insoluble polymer in the coating or layer. The weight ratio of water-insoluble to water-soluble polymers can be adjusted, for example and without limitation, from 90:10 to 10:90, from 80:20 to 20:80, from 75:25 to 25:75, from 70:30 to 30:70, from 67.5:33.5 to 33.5:67.5 from 60:40 to 40:60, from 56:44 to 44:56, or to 50:50.

The sustained release coating may also include a plasticizer such as triethyl citrate (TEC) at levels of from 3% to 50% of the combined weight of the polymers. Other additives to the coating can include titanium dioxide, talc, colloidal silicone dioxide or citric acid.

In some embodiments the dose of the adrenoceptor antagonist delivered from the sustained release formulation to a human subject is on the order of 0.5-10 mg per 1-4 hours over a dosage period of 4-10 hours. In other embodiments the dosage of the adrenoceptor antagonist in the sustained release formulation is 1-10 mg, 2-10 mg, 3-10 mg, 4-10 mg, 5-10 mg, 1-5 mg, 1-4 mg, 1-3 mg, 1-2 mg, 2-5 mg, 2-4 mg, 2-3 mg, 3-6 mg, 3-5 mg, 3-4 mg, 4-6 mg, 5-6 mg, 4-5 mg, or 5-6 mg per 1-2, 1-3, 1-4, 2-4, 3-4, or 2-3 hours. In other embodiments the sustained release formulation includes an appropriate amount of the adrenoceptor antagonist to achieve one or more of the dosage delivery strategies.

Various water-soluble polymers can be used in the disclosed formulations. Such polymers include, but are not limited to polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin or the like.

The formulations may also include one or more polymers such as an acrylic polymer, acrylic copolymer, methacrylic polymer or methacrylic copolymer, including but not limited to Eudragit® L100, Eudragit® L100-55, Eudragit®L 30 D-55, Eudragit®5100, Eudragit®4135F, Eudragit®RS, acrylic acid and methacrylic acid copolymers, methyl methacrylate, methyl methacrylate copolymers, ethoxyethyl methacrylates, cyanoethyl methacrylate, aminoalkyl methacrylate copolymer, polyacrylic acid, polymethacrylic acid, methacrylic acid alkylamine copolymer, polymethyl methacrylate, polymethacrylic acid anhydride, polymethacrylate, polyacrylamide, polymethacrylic acid anhydride and glycidyl methacrylate copolymers, an alkylcellulose such as ethylcellulose, methylcellulose, calcium carboxymethyl cellulose, certain substituted cellulose polymers such as hydroxypropyl methylcellulose phthalate, and hydroxypropyl methylcellulose acetate succinate, cellulose acetate butyrate, cellulose acetate phthalate, and cellulose acetate trimaleate, polyvinyl acetate phthalate, polyester, waxes, shellac, zein, or the like.

Eudragits are well known polymers and copolymers useful for controlled release applications. The EUDRAGIT®grades for enteric coatings are based on anionic polymers of methacrylic acid and methacrylates. They contain —COOH as a functional group. They dissolve at ranges from pH 5.5 to pH 7. EUDRAGIT®FS 30 D is the aqueous dispersion of an anionic copolymer based on methyl acrylate, methyl methacrylate and methacrylic acid. It is insoluble in acidic media, but dissolves by salt formation above pH 7.0. Eudragit L100-55 and L30-55 dissolve at pH above 5.5. Eudragit L100 and S100 dissolve at pH above 6.0.

The term effective amount of a therapeutic compound of the invention refers to the amount necessary or sufficient to realize a desired biologic effect. For example, an effective amount of a therapeutic compound associated with the invention may be that amount sufficient to ameliorate one or more symptoms of a disorder described herein, such as OSA. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compound associated with the invention without necessitating undue experimentation.

Subject doses of the compounds described herein for delivery typically range from about 0.1 μg to 10 mg per administration, which depending on the application could be given daily, weekly, or monthly and any other amount of time there between. The doses for these purposes may range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

In some embodiments a compound of the invention is administered at a dosage of between about 1 and 10 mg/kg of body weight of the mammal. In other embodiments a compound of the invention is administered at a dosage of between about 0.001 and 1 mg/kg of body weight of the mammal. In yet other embodiments a compound of the invention is administered at a dosage of between about 10-100 ng/kg, 100-500 ng/kg, 500 ng/kg-1 mg/kg, or 1-5 mg/kg of body weight of the mammal, or any individual dosage therein. In other embodiments a dosage form has 5 μg-5.3 mg of the agents described herein. In yet other embodiments a dosage form has 5.5 mg-20 mg of the agents described herein.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compound associated with the invention can be administered to a subject by any mode that delivers the therapeutic agent or compound to the desired surface, e.g., central nervous system. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, rectal and intracerebroventricular.

For oral administration, the therapeutic compounds of the invention can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the therapeutic compounds of the invention. The therapeutic agent is delivered to the lungs of a mammal while inhaling and traverses across the lung epithelial lining to the blood stream. Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of therapeutic agent. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise therapeutic agent dissolved in water at a concentration of about 0.1 to 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing therapeutic agent and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Intra-nasal delivery of a pharmaceutical composition of the present invention is also contemplated. Intra-nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The therapeutic compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a therapeutic compound of the invention optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The compositions described herein may be time-delayed formulations. A time-delayed formulation is one which may be administered in intermittent dosage forms. Such a formulation may be, for instance, a tablet-within-a-tablet formulation: a core tablet, containing the drug, surrounded by an erodible barrier. The outer barrier has a constant erosion rate that can be tuned to control the time lag prior to release of drug from the core. Multiple layers can be produced to produce the time delayed release of the drug.

Time delayed release of drugs may be also achieved by coating the active ingredients with polymers chosen to release the second and any further pulses at specific time points. This allows for the administration of a dosage form which provides a first release (pulse) drug, followed by a desired delay before a second pulse of drug. The polymers are chosen in such a way as to deliver the secondary pulses at chosen time intervals. The time intervals may be chosen based on the pharmacokinetics of the desired plasma level of the drug, and/or may be chosen based on the release site of the second pulse. By incorporating both an immediate release dosage and one or more delayed release dosages of the drug, the dosage form mimics a multiple dosing profile without repeated dosing, i.e., with only a single administration. The multiple doses may be delivered to the subject from the formulation through the course of sleep, in order to prevent the OSA.

Another example of this type of formulation is a dosage form comprising a closed capsule housing at least two drug-containing dosage units. Each dosage unit comprises two or more compressed tablets, beads, granules or particles.

The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### Examples

### Example 1: α₂-Adrenoceptor Blockade Rescues Hypoglossal Motor Activity and Neuroplasticity in Defense Against Obstructive Sleep Apnea

Atonia of pharyngeal dilator muscles secondary to decreased noradrenergic excitation of hypoglossal motoneurons during sleep is a key trigger of obstructive sleep apnea (OSA), a widespread disease for which treatment options are limited. Previous OSA drug candidates targeting various excitatory/inhibitory receptors on hypoglossal motoneurons have proved unviable in reactivating these neurons particularly during rapid-eye-movement (REM) sleep. It was discovered according to the invention that α₂-adrenergic antagonist yohimbine potently reversed the depressant effect of REM sleep on baseline hypoglossal motoneuron activity as first-line motor defense against OSA. Remarkably, yohimbine also restored the obstructive apnea-induced long-term facilitation of hypoglossal motoneuron activity (hLTF), a form of noradrenergic-dependent neuroplasticity which normally provides a second-line motor defense against OSA but was also depressed during REM sleep. Corroborating immunohistologic, optogenetic and pharmacologic evidence confirmed that yohimbine's beneficial effects on baseline hypoglossal motoneuron activity and hLTF were mediated mainly through activation of pontine A7 and A5 noradrenergic neurons. The present results suggest a two-tier mechanism of noradrenergic-dependent pathogenesis of OSA and a promising pharmacotherapy for rescuing the first- and second-line motor defenses against OSA through disinhibition of A7 and A5 neurons by α₂-adrenergic blockade.

Obstructive sleep apnea (OSA), which affects 3-7% of the general adult population and 1-4% of the pediatric population worldwide, is a leading cause of excessive daytime sleepiness and an independent risk factor for wide-ranging cardiovascular, metabolic, cognitive, and neuropsychiatric abnormalities causing decreased quality of life and life expectancy (19, 21-22). A pivotal step in the pathogenesis of OSA is sleep state-dependent atonia of upper airway dilator muscles (particularly the genioglossus, GG) causing pharyngeal collapse, an effect which is exacerbated in patients with narrowed upper airways (23). Current standard treatment using continuous positive airway pressure (CPAP) to restore upper airway patency is effective but has a poor adherence rate (24), whereas alternative treatments such as oral-appliance therapy, weight loss, upper-airway surgery and hypoglossal nerve stimulation are not always effective 20, 25).

Previous OSA drug candidates have generally targeted various excitatory receptors on hypoglossal (XII) motoneurons (HMs) supplying the GG muscle (1, 2, 29). However, related preclinical and clinical studies to date have been disappointing (26-28). Moreover, many candidate drugs have side effects such as drowsiness, weight gain and suppression of REM sleep, which are counterproductive for OSA (6, 30-32). Other drugs that target certain inhibitory receptors (such as muscarinic receptors) on HMs have well-known cardiovascular liabilities (33). Certain potassium channels expressed on HMs are thought to provide more specific drug targets for OSA but selective blockers for these inhibitory channels are presently lacking (7, 33).

Among the excitatory neuromodulators of HM activity that sets the resultant GG muscle tone, norepinephrine is the most potent (28, 34-35). However, this knowledge has yet to translate into a viable drug therapy for OSA. In a recent study, administration of a norepinephrine reuptake inhibitor desipramine in healthy subjects reportedly increased the tonic component of GG activity during non-REM sleep (36). However, a similar effect was not demonstrated during REM sleep; indeed, desipramine is known to inhibit REM sleep (37). Moreover, desipramine failed to increase the inspiratory-phasic component of GG activity, which is typically higher than the tonic component and hence, figures more prominently in protecting against OSA particularly during the inspiratory phase of the breathing rhythm—when OSA is prone to occur.

One major difficulty with a noradrenergic-based therapy for OSA is that the influence of endogenous noradrenergic drive on HM activity has proved highly complex and cannot be mimicked by direct activation of HMs with exogenous adrenergic drugs particularly during REM sleep (2, 29). In particular, recent studies reveal that obstructive apnea per se may elicit persistent facilitation of GG activity, a form of neuroplasticity (referred to as hypoglossal long-term facilitation, hLTF) which requires the activation of α₁-adrenoceptors on HMs for its induction (5). However, because hLTF is an effect rather than cause of OSA, its possible role in the pathogenesis of OSA has been poorly recognized. In theory, any hLTF resultant from obstructive apnea would serve to promptly reopen the upper airway and avert further persistence or recurrence of OSA.

In aspects of the invention it was concluded that, in order for repetitive OSA to occur, not only baseline HM activity but also hLTF (first- and second-line motor defenses against OSA, respectively) must be depressed during sleep. Further, it was discovered that therapeutic drugs targeting endogenous noradrenergic drive rather than HMs per se prove more effective in restoring baseline HM activity and hLTF in defense against OSA, especially during REM sleep.

Accordingly, the invention involves a novel drug therapy for OSA through antagonism of α₂-adrenoceptors, a set of Gᵢ protein-coupled receptors that regulates the release of norepinephrine from central noradrenergic neurons. It is demonstrated herein that the classic α₂-adrenergic blocker yohimbine effectively reversed the depressant effects of REM sleep on baseline HM activity and obstructive apnea-induced hLTF in rats, thereby rescuing these first- and second-line motor defenses against OSA. Immunohistologic, optogenetic and pharmacologic evidence is presented, indicating that induction of hLTF was mediated by episodic activation of pontine A7 and A5 noradrenergic neurons and was enhanced by blockade of α₂-adrenoceptors on these neurons. Collectively, the data demonstrate that disinhibition of A7 and A5 neurons by α₂-adrenergic blockade effectively restored baseline HM activity and obstructive apnea-induced hLTF during REM sleep, hence providing a promising drug therapy for OSA.

### Results

hLTF-Mediated Motor Defense Against Obstructive Apnea is Depressed During REM Sleep

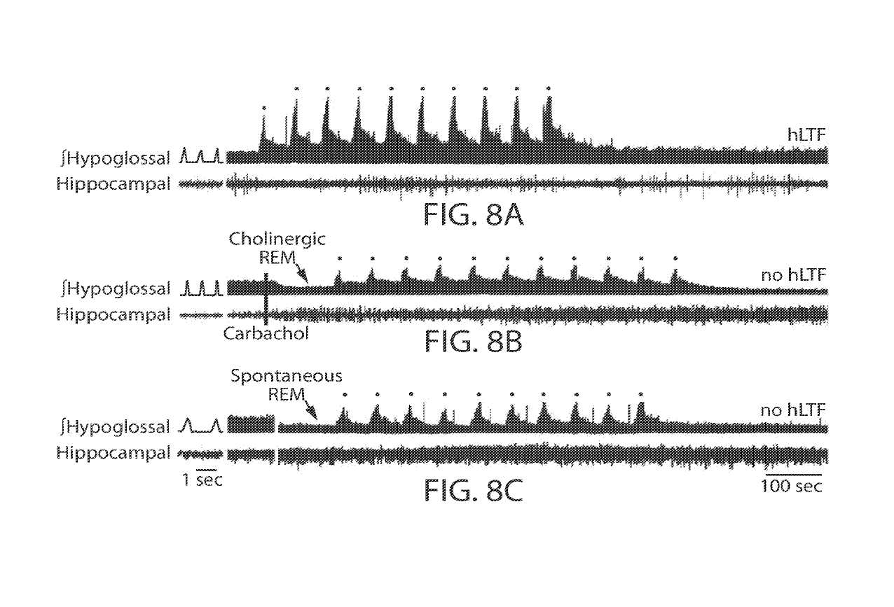

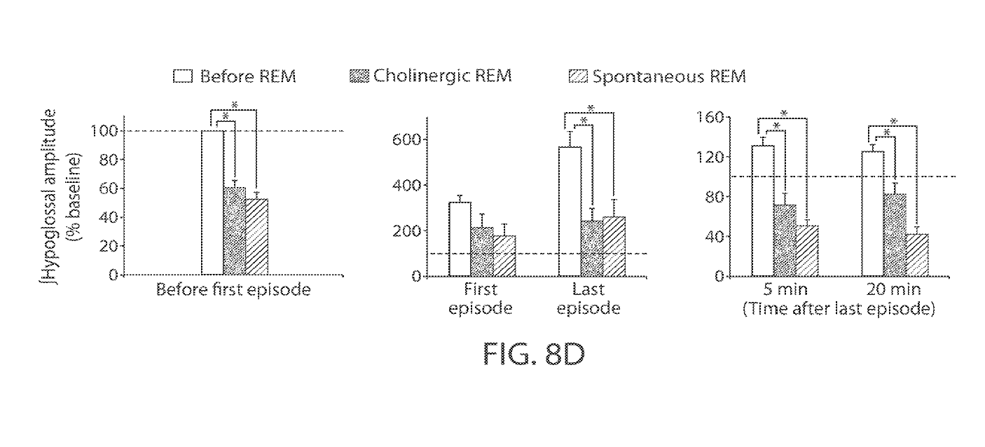

Episodic obstructive apnea simulated by repetitive artificial airway occlusion in rats (see Methodology) elicited time-dependent facilitation of HM activity (predominantly the inspiratory-phasic component) with a long-lasting memory trace afterwards evidencing hLTF (FIGS. 8A, 8D); such episodic obstructive apnea did not induce long-term facilitation of phrenic or diaphragm activity (5). To test whether obstructive apnea-induced hLTF, like baseline HM activity, was also depressed during sleep, the cholinergic agonist carbachol was injected at the pontine dorsomedial reticular formation to induce REM sleep (38-39). Cholinergic-induced REM sleep precipitated a decrease in baseline HM activity in our vagi-intact rats (n=5) (FIGS. 8B, 8D) as with vagotomized rats (1). Remarkably, throughout the period of cholinergic-induced REM sleep hLTF was also greatly attenuated, as indicated by a marked decrease in HM activity (compared with corresponding activity before REM sleep) both during the obstructive apnea episodes and long (>20 min) afterwards. To confirm that the depression of hLTF was not an artifact of the cholinergic agent per se, similar measurements were made in a separate group of rats (n=5) after transition to spontaneous REM sleep, a condition that is attainable in rats under urethane anesthesia (40). Again, spontaneous REM sleep resulted in marked decreases in not only baseline HM activity (40) but also obstructive apnea-induced hLTF (FIGS. 8C, 8D), severely blunting both these first- and second-line motor defenses against OSA.

Yohimbine Rescues Baseline HM Activity and hLTF from Depression During REM Sleep

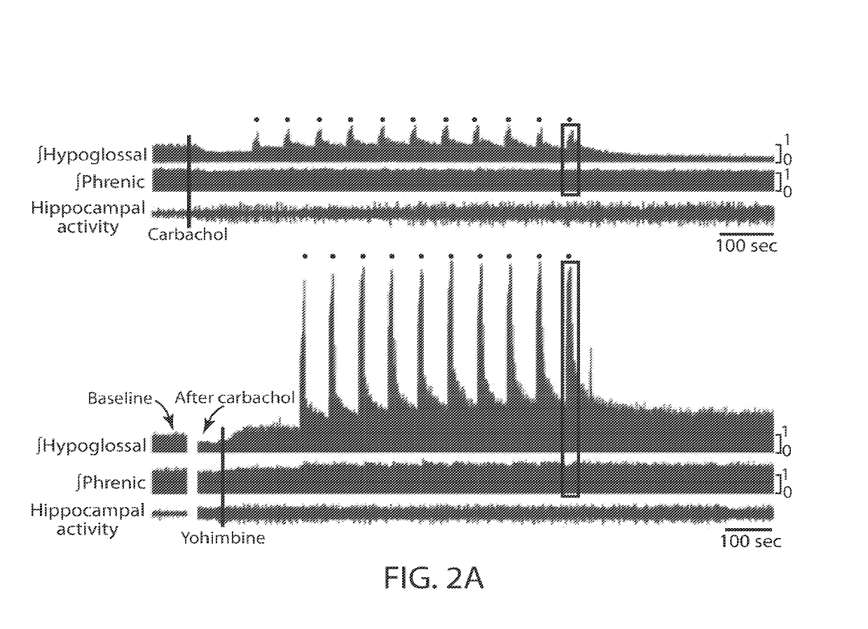

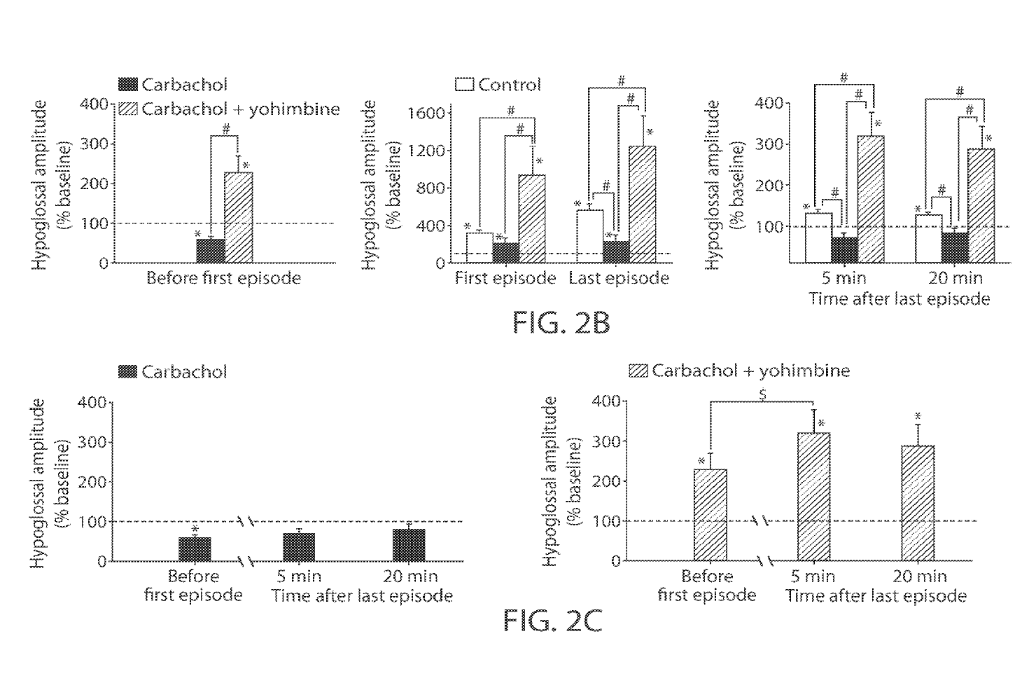

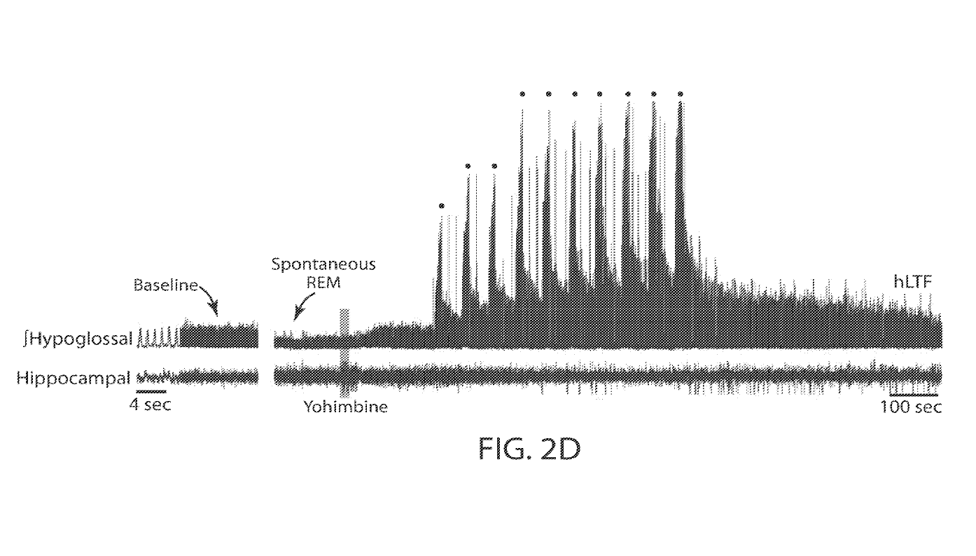

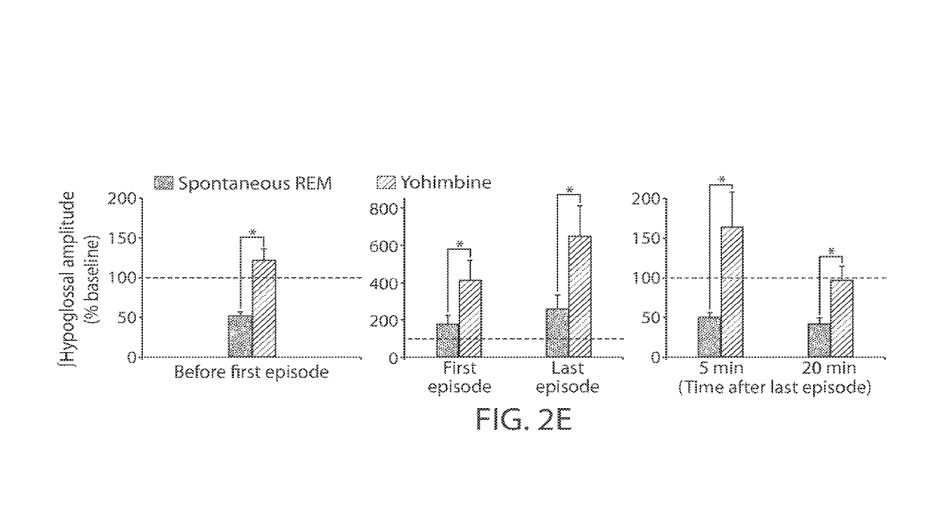

Because baseline HM activity and obstructive apnea-induced hLTF are noradrenergic-dependent (5), whether the depressions of baseline HM activity and hLTF during REM sleep could be reversed by disinhibition of central noradrenergic neurons with α₂-adrenergic blockade was tested. Indeed, systemic yohimbine (0.5-1.0 mg/kg i.v.) promptly reversed the decrease in HM activity under cholinergic-induced REM sleep, as evidenced by a resultant rise of baseline HM activity above the control level (P<0.01, n=7; FIGS. 2A-2D). In addition, systemic yohimbine also potently reversed the blunting of obstructive apnea-induced hLTF under cholinergic-induced REM sleep, with HM activity remaining well above the control level up to 20 min after episodic obstructive apnea (FIGS. 2A, 2B). Systemic yohimbine at a lower dose (0.25-0.5 mg/kg i.v.) resulted in similar therapeutic effects under spontaneous REM sleep albeit to a lesser extent presumably reflecting a dose-dependence of the therapeutic effect sizes (FIGS. 2D, 2E). Hence, yohimbine at a dose >0.25 mg/kg i.v. in rats effectively rescued both the first- and second-line motor defenses against OSA under REM sleep, when baseline HM activity was lowest.

### Pontine A7/A5 Noradrenergic Neurons are Activated by Episodic Obstructive Apnea

An emerging concept is that obstructive apnea per se may time-dependently facilitate hypoglossal activity leaving a subsequent long-lasting memory called hypoglossal long-term facilitation, a form of experience-dependent motor learning and memory which requires intermittent interruptions of vagal feedback and α₁-adrenoceptor (but not 5-HT₂ receptor) activation on hypoglossal motoneurons for its induction⁵. It was hypothesized that impairment of such experience-dependent motor defense against recurrent obstructive apneas due to noradrenergic withdrawal during sleep might contribute to the pathogenesis of OSA beyond the hypotonia of hypoglossal motoneurons. It was reasoned that reactivation of central noradrenergic drive during sleep with drug treatments that target central noradrenergic neurons rather than hypoglossal motoneurons⁶⁻⁷ could be more effective in restoring hypoglossal motoneuron excitability and hypoglossal long-term facilitation in defense against OSA.

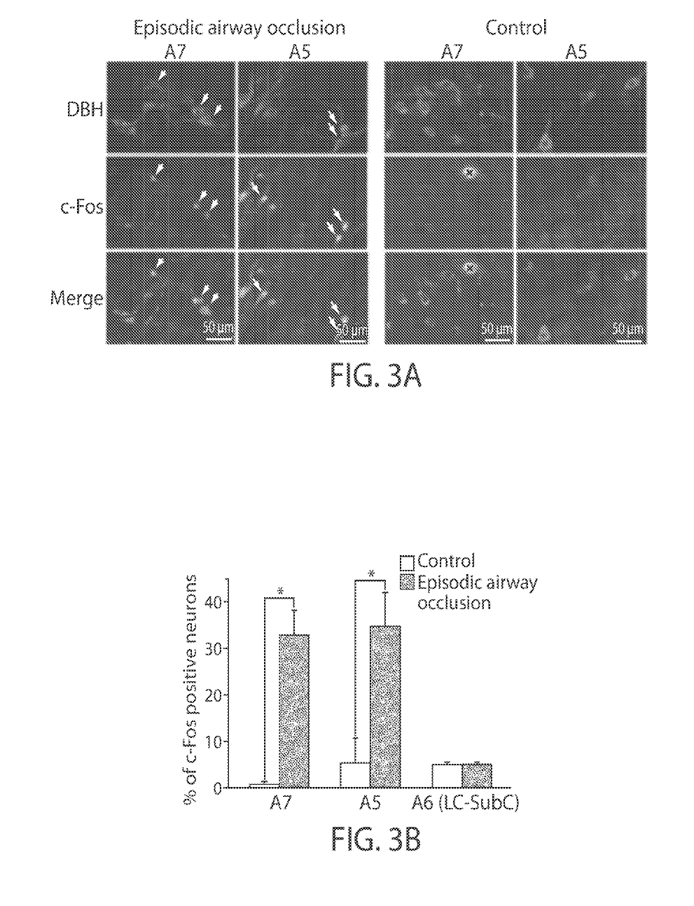

To identify the sites of action of yohimbine therapy for OSA, whether activities of pontine noradrenergic neurons known to affect breathing were influenced by obstructive apnea was investigated. In rats exposed to episodic obstructive apnea (the experimental group), c-Fos expression was markedly increased in pontine A7 and A5 neurons (identified by dopamine β-hydroxylase immunofluorescent labeling) compared with corresponding control levels indicating obstructive apnea-induced excitation of these noradrenergic neurons (FIGS. 3A, 3B). In contrast, c-Fos expressions in pontine A6 (locus coeruleus and sub-coeruleus) noradrenergic neurons were not significantly different in the experimental vs. control groups (FIG. 3B). These data suggested that induction of noradrenergic-dependent hLTF likely involved A7 and A5 neuron activation.

Episodic Optogenetic Activation of Pontine A7/A5 Noradrenergic Neurons Induces hLTF

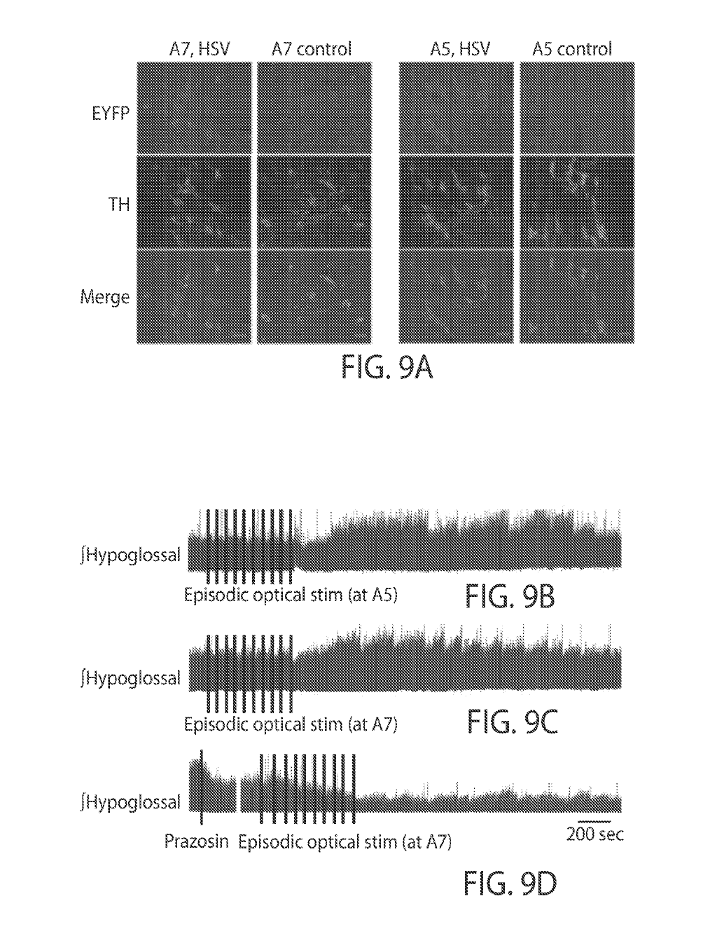

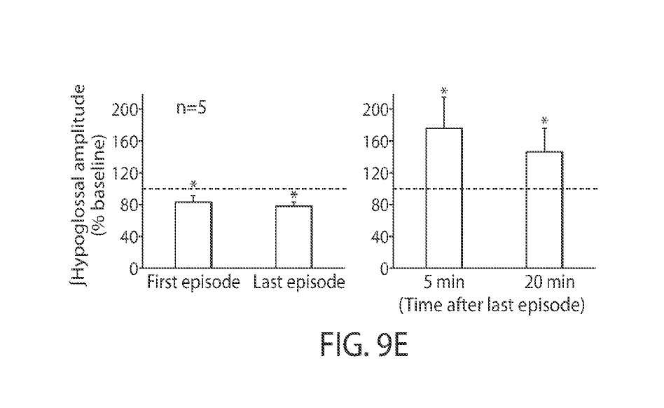

To confirm this, episodic optogenetic stimulation of A7 and A5 neurons was performed in transgenic rats which expressed Cre recombinase under the control of the endogenous tyrosine hydroxylase promoter (FIG. 9A). After 8-10 episodes of 15-sec optical stimulation of A7 or A5 neurons pretreated with a Cre-inducible viral vector encoding ChR2-EYFP (channelrhodopsin 2—enhanced yellow fluorescent protein), HM activity gradually increased to a peak at ˜5 min post-stimulation before returning to its pre-stimulation baseline level in 10-20 min (FIGS. 9B, 9C, 9E). After administration of the α₁-adrenoceptor antagonist prazosin (0.5 mg/kg, i.v.), HM activity was decreased and similar episodic optogenetic stimulation of A7 neurons no longer resulted in hLTF (FIG. 9D). Taken together, these data suggested that episodic activation of A7/A5 noradrenergic neurons could induce long-lasting facilitation of HM and GG activities.

A7/A5 Neurons Mediate Yohimbine's Beneficial Effects on Baseline HM Activity and hLTF

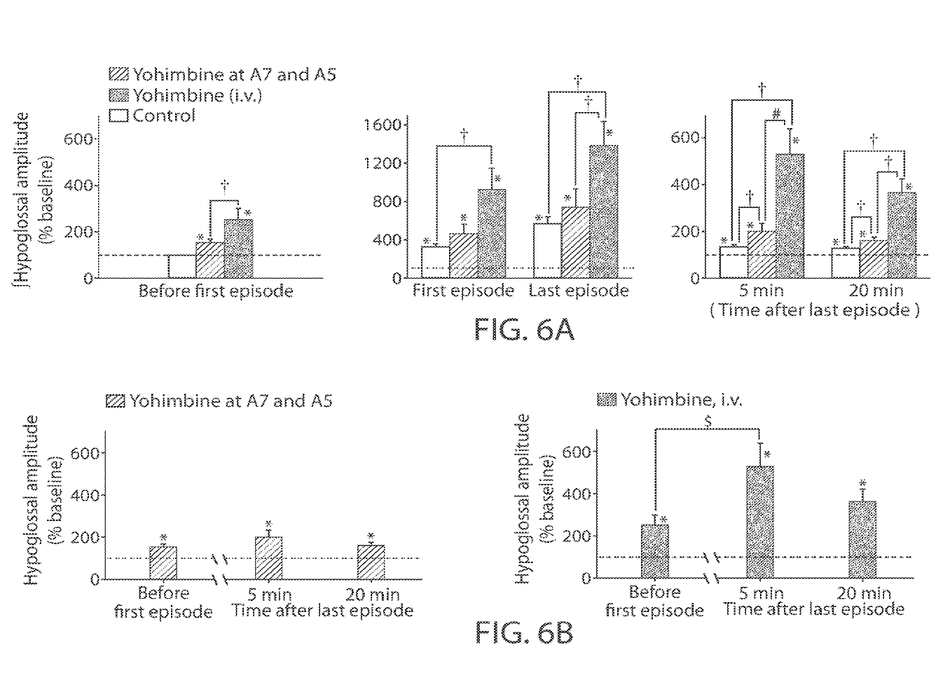

Unlike hLTF induced by episodic activation of A7 and A5 neurons with obstructive apnea (FIGS. 2, 3, 8); however, hLTF induced by direct episodic optogenetic stimulation of A7 or A5 neurons was manifested only after (but not during) stimulation (FIG. 9B, 9C, 9E). Thus, induction of hLTF by episodic obstructive apnea likely also involved other processes, such as simultaneous activations of bilateral A7 and A5 neurons and/or modulations of α₂-adrenoceptor activities therein, which could not be reproduced optogenetically. To test this, yohimbine was injected focally at bilateral A7 and A5 regions in rats before inducing hLTF with episodic obstructive apnea. In FIG. 6A, yohimbine injection at bilateral A7 and A5 regions resulted in a significant increase of baseline HM activity (P<0.01, n=5) as well as enhancement of hLTF both during the obstructive apnea episodes (P<0.05, two-way ANOVA with repeated measures) and up to 20 min afterwards (P<0.05). These beneficial effects of focal yohimbine injection at bilateral A7 and A5 regions were similar to those resulting from systemic yohimbine albeit to a lesser extent (FIG. 6A)—likely due to a more complete coverage of these and other noradrenergic regions by systemic than focal administration of yohimbine.

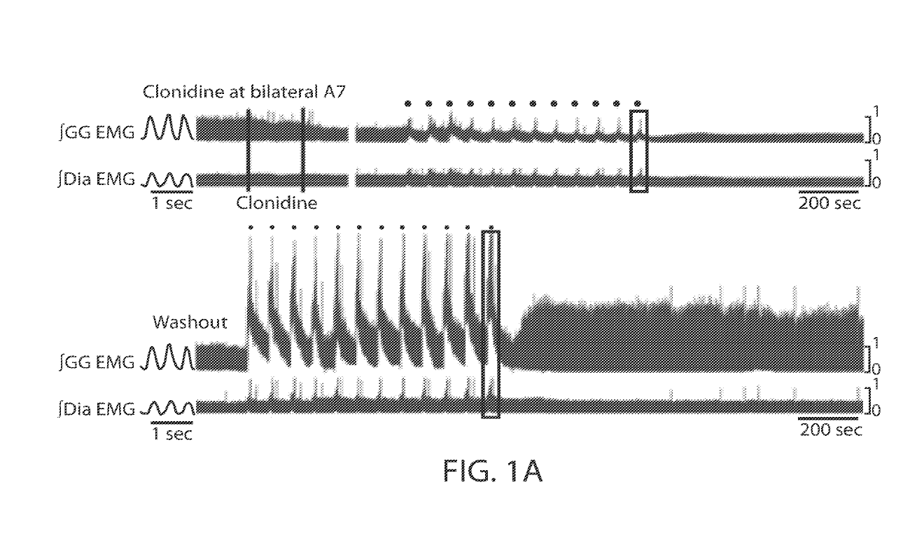

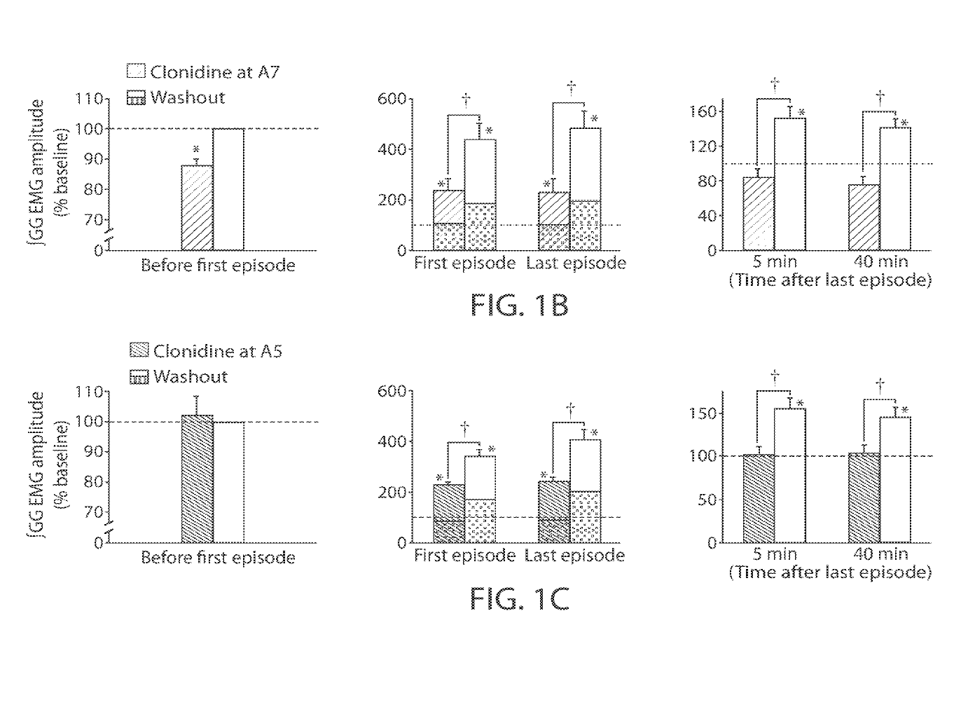

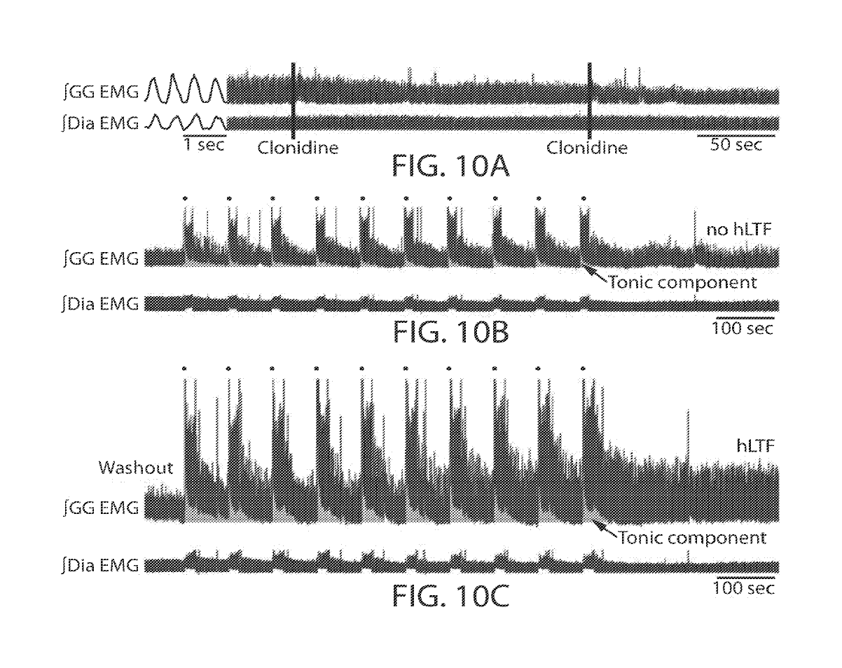

In contrast, injection of the α₂-adrenoceptor agonist clonidine at bilateral A7 region significantly reduced the inspiratory-phasic component of baseline GG EMG amplitude in our vagi-intact and spontaneously breathing rats (n=7, FIG. 10A) as with vagotomized rats (41). In addition, clonidine also markedly suppressed hLTF both during and after the obstructive apnea episodes (FIGS. 10B, 10C, 1B). Similar blunting of the obstructive apnea-induced hLTF was also observed following clonidine injection at bilateral A5 region although baseline GG activity was not significantly affected in this case (FIG. 1C). Thus, even partial withdrawal of central noradrenergic activity with relatively modest or negligible resultant decreases in GG muscle tone may severely impair the second-line motor defense against OSA mediated by hLTF of the inspiratory-phasic component of HM and GG activities during and following obstructive apnea.

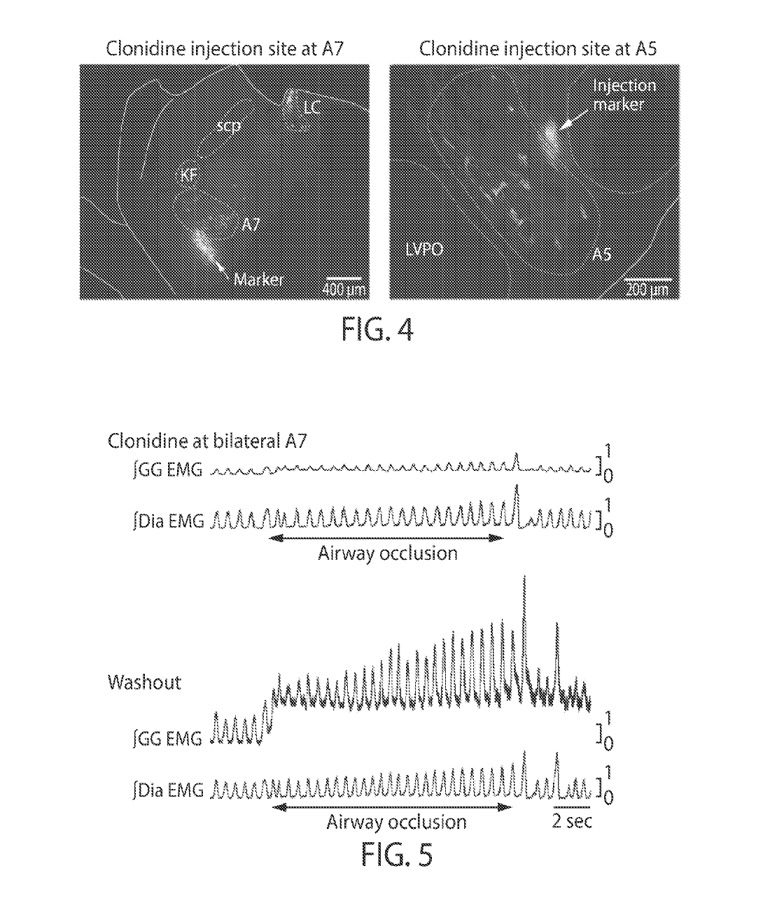

Next, A7 and A5 neuronal activity was suppressed with focal injection of the α₂-adrenoceptor selective agonist clonidine at bilateral A7 or A5 regions (FIG. 4) prior to episodic airway occlusion treatment in urethane-anesthetized spontaneously breathing rats. The presynaptic α₂-adrenoceptor on central noradrenergic neurons is a set of G, protein-coupled autoreceptors that regulates the release of norepinephrine from these neurons through negative feedback⁸⁻⁹. As expected¹⁰, injection of clonidine at bilateral A7 region reduced the integrated genioglossus EMG amplitude by ˜12% from pre-injection baseline indicating a decrease in hypoglossal activity (FIGS. 1A, 1B). Facilitation of hypoglossal activity during airway occlusion was markedly attenuated after injection of clonidine at bilateral A7 region, whereas hypoglossal long-term facilitation was totally abolished (FIGS. 1A, 1B, 5, 6). Similar blunting of obstructive apnea-induced facilitation of hypoglossal activity during and after episodic airway occlusion treatment was also observed after clonidine injection at bilateral A5 region, even though baseline genioglossus activity was not significantly reduced in this case (FIGS. 1C, 5). Thus, even partial noradrenergic withdrawal with relatively modest or negligible resultant decreases in genioglossus muscle tone may severely impair the capacity for learning and memory in the hypoglossal motor defense against airway occlusion. This observation provides new mechanistic insights as to why OSA may occur in non-REM sleep as much as in REM sleep in adult patients¹¹, despite the fact that genioglossus muscle tone is generally lowest in REM sleep^{2,12-13}.

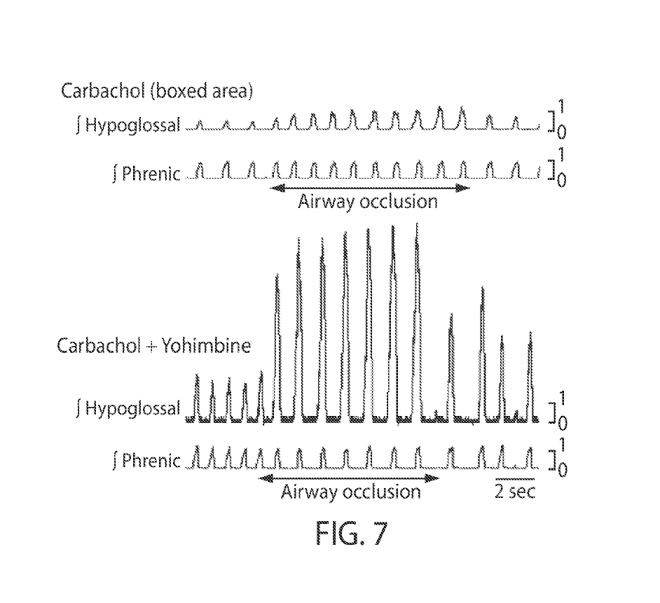

In contrast to clonidine, the α₂-adrenoceptor specific antagonist yohimbine when administered by focal injection at bilateral A7 and A5 regions or systemically by intravenous injection resulted in an up-regulation of hypoglossal motoneuron excitability and of obstructive apnea-induced hypoglossal long-term facilitation (FIG. 6). To test whether systemic yohimbine could provide an effective remedy for OSA, yohimbine was administered intravenously in urethane-anesthetized rats after focal injection of the cholinergic agonist carbachol at pontine dorsomedial reticular formation to induce a REM sleep-like state^{1,14}. Possible influence of pharyngeal negative pressure reflex on hypoglossal/genioglossus activities during airway obstruction in REM sleep¹⁵ was eliminated by subjecting the animals to positive-pressure mechanical ventilation under pancuronium bromide paralysis to suppress spontaneous breathing efforts. Transition to REM-like sleep resulted in a significant decrease (˜39%) in respiratory-related hypoglossal activity in our vagi-intact rats (FIG. 2) as in vagotomized rats¹. Importantly, facilitation of hypoglossal activity during and after episodic airway occlusion treatment was markedly attenuated throughout the period of REM-like sleep indicating impaired learning and memory capacity in the hypoglossal motor defense against airway obstruction (FIGS. 2, 7). By contrast, systemic yohimbine reversed the hypotonia of hypoglossal motoneurons during REM-like sleep, as evidenced by a resultant rise of hypoglossal activity above the pre-carbachol baseline level (FIG. 2B). Systemic yohimbine also reversed the carbachol-induced blunting of the facilitation of hypoglossal activity during and after episodic airway occlusion treatment, thereby restoring (even enhancing) the capacity for experience-dependent learning and memory in the hypoglossal motor defense against airway occlusion during REM-like sleep (FIGS. 2, 7).

Obstructive Apnea Recruits Tonic Component of GG Activity During Spontaneous Breathing

In these spontaneously breathing rats GG activity also exhibited a significant tonic component during obstructive apnea (FIG. 10C), an effect which was likely mediated by the pharyngeal negative pressure reflex (15) and was not observed before airway obstruction (FIG. 10C) or in paralyzed and mechanically ventilated rats in which airway pressure remained non-negative throughout (FIGS. 2, 8). This tonic component in the GG activity did not persist after the obstructive apnea episodes and hence, did not contribute to hLTF (FIG. 10C). As with the inspiratory-phasic component, this tonic component recruited during airway obstruction was also strongly suppressed by clonidine application at bilateral A7 (FIGS. 10B, 1B) or A5 region (FIG. 1C).

### Discussion

Rather than targeting HMs as in previous studies for potential treatment of OSA, the forgoing results demonstrate that blockade of α₂-adrenoceptors on central noradrenergic neurons effectively reversed the depression of the inspiratory-phasic component of baseline HM activity during REM sleep. Equally important, α₂-adrenoceptor antagonism also restored hLTF of the inspiratory-phasic component of HM activity in defense against obstructive apnea during REM sleep. Further, the results confirm that yohimbine's therapeutic actions on HM activity and obstructive apnea-induced hLTF were mediated mainly through blockade of α₂-adrenoceptors on A7 and A5 noradrenergic neurons. These findings provide the first proof-of-concept of α₂-adrenergic blockade as a therapy for reactivating both the first- and second-line motor defenses against OSA during sleep.

A dilemma is why direct activation of α₁-adrenoceptors on HMs did not yield similar beneficial effects under REM sleep as demonstrated here with α₂-adrenergic blockade (2, 29). The present study provides valuable insight. Specifically, the optogenetics data (FIG. 6A) show that the influence of central noradrenergic drive on HM activity is plastic (rather than reflexive), such that episodic optogenetic stimulation of A7 and A5 neurons could lead to the expression of hLTF post-stimulation. Thus, excitatory inputs to A7 and A5 neurons may interact with presynaptic α₂-adrenoceptor activity to modulate norepinephrine release in a time-dependent manner that long outlasts the duration of these inputs. Because HM activity was not increased during optogenetic stimulation of these noradrenergic neurons, induction of the post-stimulation component of hLTF did not require phasic facilitation of HM activity during stimulation of A7 or A5 neurons. Further, this finding also implies that the induction mechanism for post-stimulation hLTF was likely localized in the A7 and A5 noradrenergic pathways presynaptic to the HMs, independent of HM activity. Such episodic activity-dependent neuroplasticity in the A7 and A5 noradrenergic-HM pathways presynaptic to the HMs cannot be reproduced by sustained pharmacologic activation of α₁-adrenoceptors or other excitatory receptors on HMs.

The suggested expression of episodic activity-dependent neuroplasticity in the A7 and A5 noradrenergic-HM pathways independent of HM activity is further supported by our finding that the post-stimulation phase of obstructive apnea-induced hLTF was abolished after inhibition of A7 or A5 neurons with clonidine (FIG. 10A, 1B, 1C). Thus, integrity of both A7 and A5 groups of noradrenergic neurons was requisite for the expression of hLTF post-stimulation. Of particular significance, clonidine inhibition of A5 neurons abolished post-stimulation hLTF while baseline GG activity remained virtually unchanged (FIG. 1C). The demonstrated suppression of obstructive apnea-induced hLTF after even partial inhibition of central noradrenergic activity with minimal resultant decreases in baseline GG activity provides a possible mechanistic explanation as to why OSA is generally as prevalent in non-REM sleep as in REM sleep in adult patients (11), despite the fact that GG muscle hypotonia is typically less severe in non-REM sleep than in REM sleep (12-13). Together, the present findings support the novel concept that sleep state-dependent depression of obstructive apnea-induced hLTF may contribute importantly to the pathogenesis of OSA as much as decreases in baseline HM and GG activity per se.

Another difficulty with previous attempts of direct pharmacologic activation of HMs to counter OSA is that the resultant excitatory effects may be offset by concurrent inhibitory inputs which could be prominent particularly during REM sleep. Given that HMs are strongly inhibited during cholinergic-induced REM sleep (42) as in natural REM sleep (33, 43), it is remarkable that systemic yohimbine alone was sufficient to simultaneously disinhibit and excite HMs during both cholinergic-induced and spontaneous REM sleep (FIGS. 2A-2E). Because endogenous inhibition of HMs is generally most prominent in REM sleep when central noradrenergic activity is lowest, we expect that inhibitory inputs to HMs may be inversely gated by endogenous noradrenergic drive (or directly gated by α₂-adrenoceptor activity), perhaps in a manner similar to that seen in some brain systems (44-46). Furthermore, because central noradrenergic neurons are typically silent during REM sleep (47-48), blockade of α₂-adrenoceptors may seem superfluous as these receptors are generally presumed to be already quiescent in this state. It is therefore surprising that systemic α₂-adrenoceptor antagonism alone was sufficient to reactivate central noradrenergic drive during both cholinergic-induced and spontaneous REM sleep (FIGS. 2A-2E). It is thought that α₂-adrenoceptors on central noradrenergic neurons may be activated during REM sleep to inhibit these neurons by adrenergic (rather than noradrenergic) inputs; indeed, certain medullary adrenergic neurons (such as C1 group of neurons) are known to be active during REM sleep and exert inhibitory influence on pontine noradrenergic neurons via α₂-adrenoceptors (49-50). If so, α₂-adrenergic blockade would serve to disable such adrenergic inhibition of central noradrenergic neurons during REM sleep.

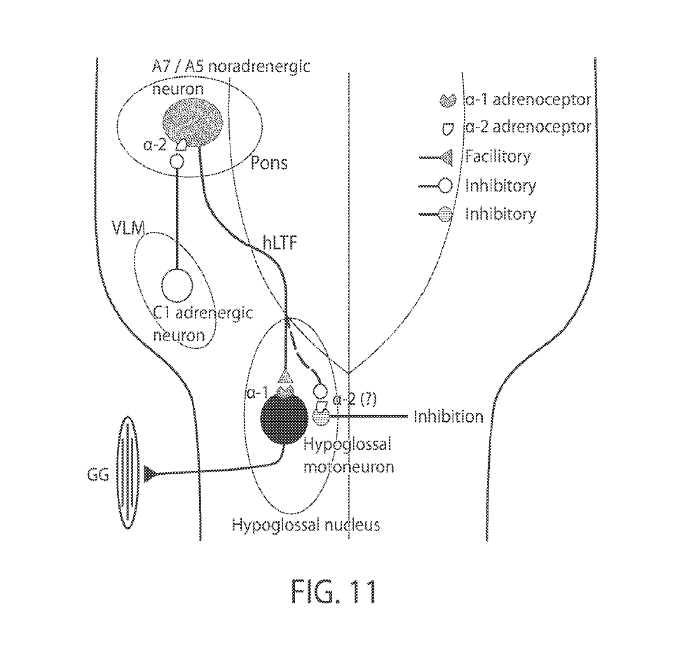

In light of the above, a two-tier mechanism of noradrenergic-dependent pathogenesis of OSA and corresponding mechanism of action of α₂-adrenoceptor antagonism therapy for OSA is proposed (FIG. 11). During REM sleep, pontine noradrenergic neurons, including A7/A5 neurons, are inhibited (via increasing α₂-adrenergic receptor activation) by inputs from C1 adrenergic neurons in medulla, which become active during REM sleep. The resultant decreases in central noradrenergic activity precipitate a fall in the inspiratory-phasic component of baseline HM activity and corresponding GG muscle tone, triggering the onset of OSA. Additionally, with decreases in A7/A5 neuronal activity during REM sleep obstructive apnea no longer induces hLTF, hence allowing the developing obstructive apnea to persist and recur. This two-tier mechanism of noradrenergic-dependent depression of the first- and second-line motor defenses against OSA underlies the pathogenesis of full-blown repetitive and unremitting OSA that can be broken only by arousal. To reverse this, administration of an α₂-adrenergic blocker, such as yohimbine, effectively removes the inhibitory influences of medullary adrenergic neurons on central noradrenergic neurons during REM sleep. Resultant reactivation of A7/A5 neurons and other central noradrenergic neurons restores the excitatory modulations of HMs by central noradrenergic drive and hLTF and simultaneously gates off inhibitory inputs to the HMs during REM sleep, thus reestablishing the first- and second-line motor defenses against OSA (FIG. 11).

Yohimbine is a prescription α₂-adrenergic blocker which has been tested extensively in acute and long-term clinical studies to verify its relative safety when administered at a clinically recommended dose (16, 51). Within this dose range yohimbine does not disrupt REM sleep (52-54) or cause weight gain (55). For comparison, a yohimbine dose of 0.5 mg/kg presently used in rats amounts to a human equivalent dose of 5.6 mg for a 70 kg person (56). Because yohimbine per se has a relatively short elimination half-life of ˜36 min in humans (57), an extended-release yohimbine formulation is desirable for effective repurposed treatment of OSA throughout sleep. For risk/benefit analysis, the off-target profile of yohimbine as a prototypic α₂-adrenergic blocker is comparable to those of traditional β₁-adrenergic blockers widely used for the treatment of hypertension and heart diseases (Table 1). In addition to the sites of action of yohimbine therapy including A7 and A5 noradrenergic neurons, therapeutic contributions of other yohimbine-sensitive receptors (in addition to α₂-adrenoceptors) expressed on these neurons may be desirable.

### Methodology

### Animal Preparation and Electrophysiology

All experimental protocols were reviewed and approved by the responsible Committee on Animal Care in accordance with published guidelines. Experiments were performed on 45 male adult Sprague-Dawley rats (300-400 g, Charles River Laboratories, Wilmington, Mass.) under urethane anesthesia (initial dose 1.5 g/kg, i.p.; supplemental dose 1/10 of initial dose, i.p. or i.v. when necessary). The femoral vein and artery were cannulated for intravenous infusion and blood pressure monitoring, respectively. Rectal temperature was maintained at 36.5-37.5° C. with a thermostatic heating pad.

After sub-laryngeal tracheal cannulation with a Y-shaped tracheal cannula, two isolated silver wires (O.D. 0.127 mm) were implanted into the genioglossus and diaphragm for EMG recording. The wire tips were exposed for 1 mm and separated by approximately 5 mm once inserted into the muscle. In some rats, the medial branch of the hypoglossal nerve (which innervates the GG and other tongue protrusion muscles) and phrenic nerve (which innervates the diaphragm) were isolated and severed. After the rat was mounted onto the stereotaxic frame, the central end of the nerve was exposed from the back of the neck (dorsal approach) and mounted onto parallel bipolar wire electrodes for recording. These rats were artificially ventilated with oxygen-enriched (40% O₂) medical air and paralyzed with pancuronium bromide.

The EMGs were amplified (CyberAmp 380, Axon Instruments, Union City), integrated (time constant 0.1 s, MA821 RSP Moving Averager, CWE) and sampled at 10 KHz into a Dell PC with LabView software (National Instruments, Austin, Tex.). The recordings were stabilized for at least 1 hour before any data collection and experimental manipulations were performed.

### Carbachol-Induced REM Sleep-Like State

A REM sleep-like state in urethane-anesthetized rats was induced by microinjection of the cholinergic agonist carbachol at unilateral dorsomedial pons^{1,14} and was detected by the decrease of hypoglossal/GG EMG activity and the appearance of hippocampal theta discharge. The latter was recorded by using a parallel bipolar wire electrode inserted into the hippocampal CA1 region at the following stereotaxic coordinates: 3.7 mm caudal from Bregma, 2.2 mm lateral from midline, and depth of 2.4 mm from brain surface.

### Brain Microinjections

For brain microinjections, an occipital craniostomy was performed to expose the brain surface. A glass micropipette (tip O.D. 10-20 μm) filled with chemical solution was inserted to the target structure according to its stereotaxic coordinates. Injections (25-50 nl per injection) were performed by applying pressure pulses to the micropipette and confirmed by the movement of the intrapipette solution meniscus. All chemicals were purchased from Sigma-Aldrich (Sigma, St. Louis, Mo.) and dissolved in ACSF at concentration of 10 mM. Stereotaxic coordinates of the A7 region: 2.3-2.6 mm from midline, 0-0.5 mm rostral from interaural level, 8.0-8.5 mm from lambda surface; A5 region: 0-0.5 mm caudal from interaural level, 2.2-2.7 mm from midline, 9.5-10 mm from lambda surface; dorsomedial pons: 1-1.5 mm from midline, 0.2-0.7 mm rostral from interaural level, 7.5-8.5 mm from lambda surface.

### Induction of Recurrent Obstructive Apneas

Recurrent obstructive apneas during spontaneous breathing in urethane-anesthetized rats were simulated by applying 10 or 12 episodes of airway occlusion at one episode per min. Each episode of airway occlusion started from the end of an expiration (in spontaneous breathing rats as described in Tadjalli et al., J Neurosci 30, 16886-16895 (2010)⁵) or lung deflation (in ventilated rats) and lasted 10-12 sec. During mechanical ventilation in urethane-anesthetized and pancuronium-paralyzed animals, recurrent obstructive apneas were simulated by periodically stopping the ventilator at end-expiration in a similar manner.

### Optical Stimulation

In rats that were preinjected with viral vectors at A7 or A5, an 0200 μm optical fiber was inserted stereotaxically into the A7 or A5 region. The optical fiber was connected to a 473-nm laser light source (IKE-PS-500, Ikecool) to deliver optical stimulation. For episodic optical stimulation, 10 square wave light pulses (each pulse lasting 15 seconds) were delivered at 1 pulse per minute for 10 minutes.

### Statistical Analyses

The amplitudes of the integrated genioglossus EMG (or hypoglossal nerve discharge) and integrated diaphragm EMG (or phrenic nerve discharge) recordings in each respiratory cycle were measured and normalized to the control baseline values. All values are expressed as mean±SE. Student t test (or one- or two-way ANOVA with repeated measures followed by Tukey post-hoc analysis, where appropriate) was used to test for statistical significance at the 95% confidence level.

### TH (Tyrosine Hydroxylase) Immunohistology

To confirm that the microinjections of clonidine and yohimbine covered the A7 and A5 groups of noradrenergic neurons, the loci of microinjections were marked by microinjections of fluorescent microspheres (RETROBEADS™, Lumafluor Inc.) at the end of the experiment. The animal was killed by urethane overdose and perfused with PBS followed by paraformaldehyde solution. The brain was removed, post-fixed and cut into 40-μm coronal sections on a freezing microtome. Sections of rostral pons were processed for immunofluorescent visualization of TH. Briefly, the sections were incubated in a rabbit polyclonal anti-TH antibody (EMD Millipore Cat# AB152, RRID: AB_390204) at 1:500 dilution in PBS containing 5% normal goat serum for 48 hours at 4° C. Then the sections were rinsed (3×15 min) and incubated in Alexa Fluor-488 labeled goat anti-rabbit IgG (Invitrogen-Life Technology, A11008) at −1:1000 dilution for 3 hours at room temperature and thoroughly rinsed.

DBH (Dopamine β-Hydroxylase) and c-Fos Immunohistology

To detect c-Fos expression in pontine noradrenergic neurons following episodic airway occlusion treatment, brainstem sections from rats (n=6) exposed to such treatments (two experimental series separated by 10 min in each rat under urethane anesthesia), brainstem sections from rats in the experimental group and in the control group(not exposed to episodic airway occlusion) were incubated in a mixture of goat polyclonal anti-dopamine β-hydroxylase (Santa Cruz Biotechnology Cat# sc-7487, RRID: AB_2230289) and rabbit polyclonal anti-c-Fos (EMD Millipore Cat# ABE457) antibodies at 1:500 dilution in PBS containing 5% normal donkey serum for 48 hours at 4° C. Then the sections were rinsed (3×15 min) and incubated in a mixture of Alexa Fluor-488 labeled donkey anti-rabbit IgG and Alexa Fluor-594 labeled donkey anti-goat IgG (A21206 and A11058 respectively, Invitrogen-Life Technology) at 1:500 dilution for 3 hours at room temperature and thoroughly rinsed.

### Fluorescent Microscopy

Brainstem sections processed for immunostaining as described above were mounted onto slides, dried and coverslipped, and observed under fluorescent microscope (Zeiss fluorescent Axio microscope, Carl Zeiss MicroImaging, LLC). Photos were captured with Axiocam (Zeiss) and analyzed with AV Rel 4.8.2 software (Zeiss).

### Optogenetics Methods

Experiments were performed on 6 homozygous TH-cre transgenic Sprague-Dawley rats (TH-cre^{tm1sage}, TGRA8400, male, 275-300 g, Horizon Discovery) that expressed Cre recombinase in catecholamine neurons under the control of the endogenous tyrosine hydroxylase (TH) promoter. Surgeries were performed under pentobarbital anesthesia (60 mg/kg, ip) and sterile condition. The first 3 rats were microinjected a cre-dependent short-term HSV vector encoding ChR2-EYFP (HSV-LS1L-hChR2(H134R)-EYFP) at unilateral A7 (n=2) or A5 (n=1). The dosage was 2 μl at viral concentration ≥3×10⁸ transducing units/ml. This vector used nonleaky lox-stop-lox cassette that was induced robustly in the presence of cre to drive the expression of ChR2-EYFP for a short period of 3-7 days. The other 3 rats were microinjected a cre-dependent AAV vector encoding ChR2-EYFP (AAV9-hEF1a-DIO-hChR2(H134R)-EYFP) at unilateral A7. The dosage was 2 μl at viral concentration 2.22×10¹³ transducing units/ml. After the injection, rats were maintained under standard post-surgical care until the ChR2-EYFP transduction was complete (4-5 days for rats microinjected HSV vector and 4 weeks for rats microinjected AAV vector) before being subjected to optical stimulation experiments.

### Example 2: Preclinical Study of BRL44408 Treatment of Obstructive Sleep Apnea

Obstructive sleep apnea (OSA) is caused by a decrease of hypoglossal motoneuron activity during sleep and resultant loss of upper airway dilator muscle tone. In a preclinical study in rats we found that the decrease of hypoglossal activity during spontaneous REM sleep was effectively reversed by BRL44408, a selective antagonist for adrenergic α2A receptor.

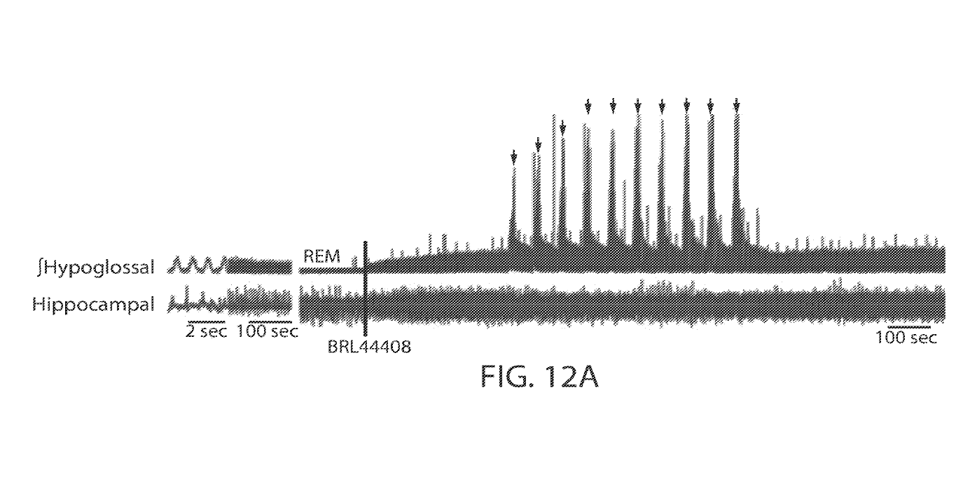

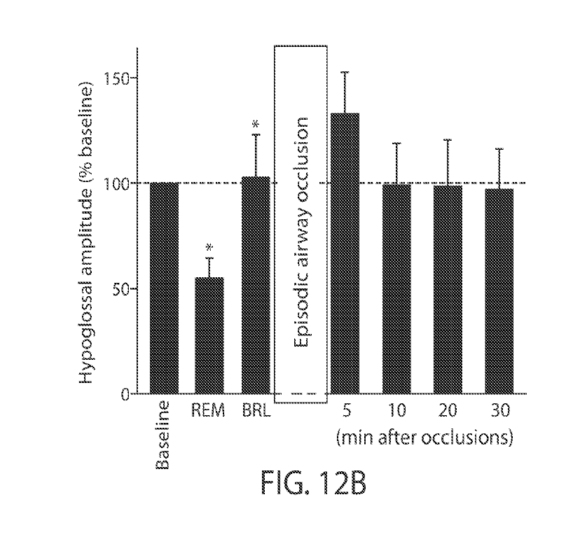

Experiments were performed on 5 adult male rats (body weight 270-550 g) under urethane anesthesia, pancuronium paralysis and artificial ventilation. Unlike other anesthetics, urethane anesthesia retains the sleep-like alternations in brain state and in breathing pattern and chemosensitivity similar to those seen in natural sleep⁵⁸⁻⁵⁹ (including state-dependent modulations of GG and abdominal respiratory-related activities⁴⁰), while also circumventing the highly fragmented natural sleep pattern in rodents⁴⁰. As shown in FIGS. 12A-12B, during periods of REM-like sleep (as indicated by increased hippocampal activity) hypoglossal nerve discharge was markedly depressed (by 44.8±9.1%, P<0.01). Intravenous application of adrenergic α-2A antagonist BRL44408 (0.2 mg/kg) effectively restored the hypoglossal discharge to normal baseline level. In addition, episodic end-expiratory airway occlusion (lasting 15 seconds each, 1 episode per minute; FIG. 12A, arrows) caused marked time-dependent increases in the amplitude of hypoglossal nerve discharge during each airway occlusion episode. Throughout spontaneous REM sleep, BRL44408 treatment continued to maintain hypoglossal activity at or above normal baseline level before, during or after episodic airway occlusion.

Results show that BRL44408 provided an effective treatment of OSA in restoring hypoglossal activity during REM sleep.

### REFERENCES

1. Fenik, V. B., Davies, R. O. & Kubin, L. REM sleep-like atonia of hypoglossal (XII) motoneurons is caused by loss of noradrenergic and serotonergic inputs. American journal of respiratory and critical care medicine 172, 1322-1330 (2005).
2. Chan, E., Steenland, H. W., Liu, H. & Horner, R. L. Endogenous excitatory drive modulating respiratory muscle activity across sleep-wake states. American journal of respiratory and critical care medicine 174, 1264-1273 (2006).
3. White, D. P. The pathogenesis of obstructive sleep apnea: advances in the past 100 years. Am J Respir Cell Mol Biol 34, 1-6 (2006).
4. Jelev, A., Sood, S., Liu, H., Nolan, P. & Horner, R. L. Microdialysis perfusion of 5-HT into hypoglossal motor nucleus differentially modulates genioglossus activity across natural sleep-wake states in rats. J Physiol 532, 467-481 (2001).
5. Tadjalli, A., Duffin, J. & Peever, J. Identification of a novel form of noradrenergic-dependent respiratory motor plasticity triggered by vagal feedback. J Neurosci 30, 16886-16895 (2010).
6. Veasey, S. & White, D. P. Obstructive sleep apnea pharmacotherapy: one step closer. American journal of respiratory and critical care medicine 187, 226-227 (2013).
7. Grace, K. P., Hughes, S. W. & Horner, R. L. Identification of a pharmacological target for genioglossus reactivation throughout sleep. Sleep 37, 41-50 (2014).
8. Hein, L., Altman, J. D. & Kobilka, B. K. Two functionally distinct alpha2-adrenergic receptors regulate sympathetic neurotransmission. Nature 402, 181-184 (1999).
9. Washburn, M. & Moises, H. C. Electrophysiological correlates of presynaptic alpha 2-receptor-mediated inhibition of norepinephrine release at locus coeruleus synapses in dentate gyrus. J Neurosci 9, 2131-2140 (1989).
10. Fenik, V. B., Rukhadze, I. & Kubin, L. Inhibition of pontine noradrenergic A7 cells reduces hypoglossal nerve activity in rats. Neuroscience 157, 473-482 (2008).
11. Siddiqui, F., Walters, A. S., Goldstein, D., Lahey, M. & Desai, H. Half of patients with obstructive sleep apnea have a higher NREM AHI than REM AHI. Sleep Med 7, 281-285 (2006).
12. Fung, S. J. & Chase, M. H. Control of hypoglossal motoneurones during naturally occurring sleep and wakefulness in the intact, unanaesthetized cat: a field potential study. J Sleep Res 23, 469-474 (2014).
13. McSharry, D. G., Saboisky, J. P., Deyoung, P., Jordan, A. S., Trinder, J., Smales, E., Hess, L., Chamberlin, N. L. & Malhotra, A. Physiological mechanisms of upper airway hypotonia during REM sleep. Sleep 37, 561-569 (2014).
14. Fenik, V. B. & Kubin, L. Differential localization of carbachol- and bicuculline-sensitive pontine sites for eliciting REM sleep-like effects in anesthetized rats. J Sleep Res 18, 99-112 (2009).
15. Shea, S. A., Edwards, J. K. & White, D. P. Effect of wake-sleep transitions and rapid eye movement sleep on pharyngeal muscle response to negative pressure in humans. J Physiol 520 Pt 3, 897-908 (1999).
16. Tam, S. W., Worcel, M. & Wyllie, M. Yohimbine: a clinical review. Pharmacol Ther 91, 215-243 (2001).
17. Burgess, C. R. & Peever, J. H. A noradrenergic mechanism functions to couple motor behavior with arousal state. Curr Biol 23, 1719-1725 (2013).
18. Philipp, M., Brede, M. & Hein, L. Physiological significance of alpha(2)-adrenergic receptor subtype diversity: one receptor is not enough. Am J Physiol Regul Integr Comp Physiol 283, R287-295 (2002).
19. Jordan, A. S., McSharry, D. G. & Malhotra, A. Adult obstructive sleep apnoea. Lancet 383, 736-747 (2014).
20. Strollo, P. J., Jr., Soose, R. J., Maurer, J. T., de Vries, N., Cornelius, J., Froymovich, O., Hanson, R. D., Padhya, T. A., Steward, D. L., Gillespie, M. B., Woodson, B. T., Van de Heyning, P. H., Goetting, M. G., Vanderveken, O. M., Feldman, N., Knaack, L. & Strohl, K. P. Upper-airway stimulation for obstructive sleep apnea. N Engl J Med 370, 139-149 (2014).
21. T. Young, P. E. Peppard, D. J. Gottlieb, Epidemiology of obstructive sleep apnea: a population health perspective. American journal of respiratory and critical care medicine 165, 1217-1239 (2002).
22. N. M. Punjabi, B. S. Caffo, J. L. Goodwin, D. J. Gottlieb, A. B. Newman, G. T. O'Connor, D. M. Rapoport, S. Redline, H. E. Resnick, J. A. Robbins, E. Shahar, M. L. Unruh, J. M. Samet, Sleep-disordered breathing and mortality: a prospective cohort study. PLoS Med 6, e1000132 (2009).
23. D. P. White, Pathogenesis of obstructive and central sleep apnea. American journal of respiratory and critical care medicine 172, 1363-1370 (2005).
24. T. E. Weaver, R. R. Grunstein, Adherence to continuous positive airway pressure therapy: the challenge to effective treatment. Proc Am Thorac Soc 5, 173-178 (2008).
25. A. Qaseem, J. E. Holty, D. K. Owens, P. Dallas, M. Starkey, P. Shekelle, Management of obstructive sleep apnea in adults: A clinical practice guideline from the American College of Physicians. Ann Intern Med 159, 471-483 (2013).
26. C. M. Lin, Y. S. Huang, C. Guilleminault, Pharmacotherapy of obstructive sleep apnea. Expert Opin Pharmacother 13, 841-857 (2012).
27. M. Mason, E. J. Welsh, I. Smith, Drug therapy for obstructive sleep apnoea in adults. Cochrane Database Syst Rev 5, CD003002 (2013).
28. R. L. Homer, Neuromodulation of hypoglossal motoneurons during sleep. Respir Physiol Neurobiol 164, 179-196 (2008).
29. P. C. Neuzeret, K. Sakai, F. Gormand, T. Petitjean, C. Buda, J. P. Sastre, S. Parrot, G. Guidon, J. S. Lin, Application of histamine or serotonin to the hypoglossal nucleus increases genioglossus muscle activity across the wake-sleep cycle. J Sleep Res 18, 113-121 (2009).
30. F. G. Issa, Effect of clonidine in obstructive sleep apnea. Am Rev Respir Dis 145, 435-439 (1992).
31. D. W. Carley, C. Olopade, G. S. Ruigt, M. Radulovacki, Efficacy of mirtazapine in obstructive sleep apnea syndrome. Sleep 30, 35-41 (2007).
32. N. S. Marshall, B. J. Yee, A. V. Desai, P. R. Buchanan, K. K. Wong, R. Crompton, K. L. Melehan, N. Zack, S. G. Rao, R. M. Gendreau, J. Kranzler, R. R. Grunstein, Two randomized placebo-controlled trials to evaluate the efficacy and tolerability of mirtazapine for the treatment of obstructive sleep apnea. Sleep 31, 824-831 (2008).
33. K. P. Grace, S. W. Hughes, R. L. Horner, Identification of the mechanism mediating genioglossus muscle suppression in REM sleep. American journal of respiratory and critical care medicine 187, 311-319 (2013).
34. L. Kubin, Sleep-wake control of the upper airway by noradrenergic neurons, with and without intermittent hypoxia. Prog Brain Res 209, 255-274 (2014).
35. G. D. Funk, J. D. Zwicker, R. Selvaratnam, D. M. Robinson, Noradrenergic modulation of hypoglossal motoneuron excitability: developmental and putative state-dependent mechanisms. Arch Ital Biol 149, 426-453 (2011).
36. L. Taranto-Montemurro, B. A. Edwards, S. A. Sands, M. Marques, D. J. Eckert, D. P. White, A. Wellman, Desipramine increases genioglossus activity and reduces upper airway collapsibility during non-REM sleep in healthy subjects. American journal of respiratory and critical care medicine PMID: 26967681, (2016).
37. R. J. Ross, P. J. Gresch, W. A. Ball, L. D. Sanford, A. R. Morrison, REM sleep inhibition by desipramine: evidence for an alpha-1 adrenergic mechanism. Brain Res 701, 129-134 (1995).
38. R. L. Homer, L. Kubin, Pontine carbachol elicits multiple rapid eye movement sleep-like neural events in urethane-anaesthetized rats. Neuroscience 93, 215-226 (1999).
39. C. J. Van Dort, D. P. Zachs, J. D. Kenny, S. Zheng, R. R. Goldblum, N. A. Gelwan, D. M. Ramos, M. A. Nolan, K. Wang, F. J. Weng, Y. Lin, M. A. Wilson, E. N. Brown, Optogenetic activation of cholinergic neurons in the PPT or LDT induces REM sleep. Proc Natl Acad Sci USA 112, 584-589 (2015).
40. S. Pagliardini, J. J. Greer, G. D. Funk, C. T. Dickson, State-dependent modulation of breathing in urethane-anesthetized rats. J Neurosci 32, 11259-11270 (2012).
41. V. B. Fenik, I. Rukhadze, L. Kubin, Inhibition of pontine noradrenergic A7 cells reduces hypoglossal nerve activity in rats. Neuroscience 157, 473-482 (2008).
42. J. Yamuy, S. J. Fung, M. Xi, F. R. Morales, M. H. Chase, Hypoglossal motoneurons are postsynaptically inhibited during carbachol-induced rapid eye movement sleep. Neuroscience 94, 11-15 (1999).
43. S. J. Fung, M. H. Chase, Postsynaptic inhibition of hypoglossal motoneurons produces atonia of the genioglossal muscle during rapid eye movement sleep. Sleep 38, 139-146 (2015).
44. S. D. Merrywest, H. Fischer, K. T. Sillar, Alpha-adrenoreceptor activation modulates swimming via glycinergic and GABAergic inhibitory pathways in Xenopus laevis tadpoles. Eur J Neurosci 15, 375-383 (2002).
45. S. P. Kuo, L. O. Trussell, Spontaneous spiking and synaptic depression underlie noradrenergic control of feed-forward inhibition. Neuron 71, 306-318 (2011).
46. S. Kondo, A. Marty, Differential effects of noradrenaline on evoked, spontaneous and miniature IPSCs in rat cerebellar stellate cells. J Physiol 509 (Pt 1), 233-243 (1998).
47. V. Fenik, V. Marchenko, P. Janssen, R. O. Davies, L. Kubin, A5 cells are silenced when REM sleep-like signs are elicited by pontine carbachol. J Appl Physiol (1985) 93, 1448-1456 (2002).
48. L. Leger, R. Goutagny, E. Sapin, D. Salvert, P. Fort, P. H. Luppi, Noradrenergic neurons expressing Fos during waking and paradoxical sleep deprivation in the rat. J Chem Neuroanat 37, 149-157 (2009).
49. G. Aston-Jones, B. Astier, M. Ennis, Inhibition of noradrenergic locus coeruleus neurons by C1 adrenergic cells in the rostral ventral medulla. Neuroscience 48, 371-381 (1992).
50. G. M. Stettner, Y. Lei, K. Benincasa Herr, L. Kubin, Evidence that adrenergic ventrolateral medullary cells are activated whereas precerebellar lateral reticular nucleus neurons are suppressed during REM sleep. PLoS One 8, e62410 (2013).
51. E. Ernst, M. H. Pittler, Yohimbine for erectile dysfunction: a systematic review and meta-analysis of randomized clinical trials. J Urol 159, 433-436 (1998).
52. A. Gentili, M. F. Godschalk, D. Gheorghiu, K. Nelson, D. A. Julius, T. Mulligan, Effect of clonidine and yohimbine on sleep in healthy men: a double-blind, randomized, controlled trial. Eur J Clin Pharmacol 50, 463-465 (1996).
53. A. Autret, M. Minz, T. Beillevaire, H. P. Cathala, H. Schmitt, Effect of clonidine on sleep patterns in man. Eur J Clin Pharmacol 12, 319-322 (1977).
54. O. Kanno, P. Clarenbach, Effect of clonidine and yohimbine on sleep in man: polygraphic study and EEG analysis by normalized slope descriptors. Electroencephalogr Clin Neurophysiol 60, 478-484 (1985).
55. N. Cimolai, T. Cimolai, Yohimbine use for physical enhancement and its potential toxicity. J Diet Suppl 8, 346-354 (2011).
56. Center for Drug Evaluation and Research, Guidance for Industry: Estimating the Maximum Safe Starting Dose in Initial Clinical Trials for Therapeutics in Adult Healthy Volunteers. (Food and Drug Administration, U. S. Department of Health and Human Services, 2005).
57. J. A. Owen, S. L. Nakatsu, J. Fenemore, M. Condra, D. H. Surridge, A. Morales, The pharmacokinetics of yohimbine in man. Eur J Clin Pharmacol 32, 577-582 (1987).
58. J. A. Boon, N. B. Garnett, J. M. Bentley, W. K. Milsom, Respiratory chemoreflexes and effects of cortical activation state in urethane anesthetized rats. Respir Physiol Neurobiol 140, 243-256 (2004).
59. E. A. Clement, A. Richard, M. Thwaites, J. Ailon, S. Peters, C. T. Dickson, Cyclic and sleep-like spontaneous alternations of brain state under urethane anaesthesia. PLoS ONE 3, e2004 (2008).
60. B. Mokhlesi, N. M. Punjabi, “REM-related” obstructive sleep apnea: an epiphenomenon or a clinically important entity? Sleep 35, 5-7 (2012).

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. A method for treating obstructive sleep apnea (OSA) comprising
administering to a subject having OSA an agent for promoting hypoglossal motoneuron excitability in an effective amount to treat OSA, optionally, wherein the agent for promoting hypoglossal motoneuron excitability restores experience-dependent hypoglossal motor learning and memory capacity.

2. A composition for use in a method for treating obstructive sleep apnea (OSA) comprising an agent for promoting hypoglossal motoneuron excitability in an effective amount to treat OSA.

3. The method of claim 1, wherein the agent for promoting hypoglossal motoneuron excitability is a disinhibitor of central noradrenergic neurons.

4. The method of claim 1, wherein the agent for promoting hypoglossal motoneuron excitability is a stimulant of central noradrenergic neurons.

5. The method of claim 3, wherein the disinhibitor of central noradrenergic neurons is an α₂-adrenoceptor antagonist.

6. The method of claim 5, wherein the disinhibitor of central noradrenergic neurons is an α₂-adrenoceptor alpha-2A antagonist.

7. The method of claim 5, wherein the disinhibitor of central noradrenergic neurons is an α₂-adrenoceptor alpha-2C antagonist.

8. The method of claim 5, wherein the α₂-adrenoceptor antagonist is selected from the group consisting of a yohimbine, BRL44408, Atipamezole, MK-912, RS-79948, RX 821002, [3H]2-methoxy-idazoxan, and JP-1302.

9. The method of claim 8, wherein the α₂-adrenoceptor antagonist is BRL44408.

10. The method of claim 8, wherein the yohimbine is a purified yohimbine pharmaceutical formulated in a sustained release formulation.

11. The method of claim 1, wherein the agent for promoting hypoglossal motoneuron excitability is in a sustained release formulation.

12. The method of claim 11, wherein the sustained release formulation is designed to release the agent over 6-10 hours.

13. The method of claim 11, wherein the sustained release formulation is designed to release the agent over 8 hours.

14. The method of claim 1, wherein the agent for promoting hypoglossal motoneuron excitability is administered to the subject before bedtime.

15. The method of claim 1, wherein the agent for promoting hypoglossal motoneuron excitability acts directly on an A7 and/or A5 neuron.

16. The method of claim 1, wherein the agent for promoting hypoglossal motoneuron excitability acts upstream of an A7 and/or A5 neuron by inducing an activator of the A7 and/or A5 neuron.

17. The method of claim 1, wherein the agent for promoting hypoglossal motoneuron excitability acts upstream of an A7 and/or A5 neuron by blocking an inhibitor of the A7 and/or A5 neuron.

18. The method of claim 1, wherein the agent for promoting hypoglossal motoneuron excitability is administered to the subject on a daily basis for 6 months.

19. The method of claim 1, wherein the agent for promoting hypoglossal motoneuron excitability is administered to the subject on a daily basis for 3 months.

20. The method of claim 1, wherein the subject has been diagnosed with OSA and is selected for treatment on the basis of the diagnosis of OSA.

21. The method of claim 1, wherein the agent for promoting hypoglossal motoneuron excitability is administered to the subject on a daily basis in several cycles, wherein the subject is not administered the agent for promoting hypoglossal motoneuron excitability for a period of time in between cycles.

22. The method of claim 1, wherein the subject is not coadministered a serotonin receptor antagonist with the agent for promoting hypoglossal motoneuron excitability.

23-29. (canceled)

30. A composition comprising a sustained release tablet or capsule comprising an agent for promoting hypoglossal motoneuron excitability and one or more sustained release coatings constructed arranged to release the agent for promoting hypoglossal motoneuron excitability over a period of 6-10 hours.

31. The composition of claim 30, wherein the sustained release tablet or capsule is constructed and arranged to release the agent for promoting hypoglossal motoneuron excitability in a dosage of 3-6 mg per 1-3 hours.

32. The composition of claim 30, wherein the sustained release tablet or capsule is constructed and arranged to release the agent for promoting hypoglossal motoneuron excitability during REM and nonREM sleep.

33. The composition of claim 30, wherein the agent for promoting hypoglossal motoneuron excitability is an α2-adrenoceptor antagonist.

34. The composition of claim 30, wherein the agent for promoting hypoglossal motoneuron excitability is an α2-adrenoceptor alpha-2A antagonist.

35. The composition of claim 30, wherein the agent for promoting hypoglossal motoneuron excitability is an α2-adrenoceptor alpha-2C antagonist.

36. The composition of claim 33, wherein the α2-adrenoceptor antagonist is selected from the group consisting of a yohimbine, BRL44408, Atipamezole, MK-912, RS-79948, RX 821002, [3H]2-methoxy-idazoxan, and JP-1302.

37. The composition of claim 36, wherein the yohimbine is derived from a yohimbe herbal extract.

38. The composition of claim 30, wherein the composition does not include a serotonin receptor antagonist.
